(19) United States
(12) Patent Application Publication
(10) Pub. No.: US 2024/0352475 A1
(43) Pub. Date: Oct. 24, 2024

(54) HIGH EXPRESSION OF ANIMAL HEME PROTEIN IN PLANTS

(71) Applicant: MOOLEC SCIENCE LIMITED, Warwick (GB)

(72) Inventors: Gaston PALADINI, Rosario (AR); Martin SALINAS, Funes (AR); Amit DHINGRA, College Station, TX (US); Henk HOOGENKAMP, Molenhoek (NL); Bruce Williamson BENAVIDES, Moscow, ID (US); Maria Laura MALVINO, Camarillo, CA (US); Balaji VASUDEVAN, Apex, NC (US)

(21) Appl. No.: 18/647,942

(22) Filed: Apr. 26, 2024

Related U.S. Application Data
(63) Continuation of application No. PCT/IB2023/056287, filed on Jun. 16, 2023.
(60) Provisional application No. 63/367,299, filed on Jun. 29, 2022.

(51) Int. Cl. C12N 15/82; A23J 3/04; A23J 3/22; C07K 14/805

(52) U.S. Cl. CPC C12N 15/8257; A23J 3/04; A23J 3/227; C07K 14/805; C12N 15/8234

(57) ABSTRACT

The present disclosure provides methods of producing heme proteins in transgenic plants, plant tissues, or plant cells, as well as describing the expression of these heme proteins in seeds. Also, the present disclosure provides transgenic plants expressing the heme proteins, myoglobin and hemoglobin, by introducing and integrating the recombinant DNA constructs into the host genetic material of the subject plants. The specific combination of regulatory elements disclosed herein allows for high heme protein expression level in seeds.

### CROSS REFERENCE TO RELATED APPLICATIONS

This application is a Continuation of PCT/IB2023/056287, filed Jun. 16, 2023, which also claims the priority benefit of U.S. Provisional Application No. 63/367,299, filed Jun. 29, 2022, which are hereby incorporated by reference in their entirety.

### REFERENCE TO SEQUENCE LISTING SUBMITTED ELECTRONICALLY

The content of the electronically submitted sequence listing in XML format (Name: 5061_0020002_Seqlisting_ST26.xml; Size: 43,209 bytes; and Date of Creation: Apr. 15, 2024), filed with the application, is incorporated herein by reference in its entirety.

### FIELD OF DISCLOSURE

The present disclosure relates to the production of animal heme proteins in transgenic plants. The present disclosure is also related to food compositions comprising recombinant heme proteins produced in genetically engineered plants. The present disclosure also relates to improved expression cassettes for the production of animal heme proteins in transgenic plants and transgenic seeds comprising selected regulatory elements and codon-optimized protein coding sequences which result in substantially high (e.g. >5%, >8%, and >10% total soluble protein [tsp]) expression levels of recombinant proteins in plant seeds.

### BACKGROUND

Climate change as well as the expected global population growth to 9.7 billion by 2050 are demanding more sustainable lifestyle practices. Livestock production supplies most of the dietary protein; however, livestock causes about 18% of the global greenhouse gas emissions (Stehfest et al. (2009), Clim. Change 95:83). These greenhouse emissions caused by livestock production are predicted to increase by 80% by 2050 (Tilman & Clark, (2014), Nature 515:518).

Meat production suffers from other problems, such as high resource intake, presence of antibiotic residues in the meat, zoonotic diseases, and ethical concerns related to exploiting animals. Public health issues, such as type 2 diabetes, cardiovascular disease, and cancer, are also associated with meat consumption (Zhang et al. (2022), Curr. Opin. Food Sci. 43:43). Despite these problems, meat has a special status in human diet and continues an unprecedented rise in demand. During the last two decades, there has been a 58% increase in global demand for meat. By 2050, studies project an increase of 62-144% in total meat consumption (Alexandratos & Bruinsma, (2012), Agricultural Development Economics Division; Valin et al (2014), J. Agric. Econ. 45:51).

Proposed mitigation efforts to livestock production and consumption include a shift to a plant-based protein diet. The demand for plant-based protein is on the rise due to its health benefits, environmentally friendly production, animal welfare, as well a consumer taste-based curiosity (Johansson, (2019) Master's thesis Chalmers University of Technology; World Health Organization, (2015). wvww.who.int/en/news-room/fact-sheets/detail/obesity-and-overweight). A review of 91 articles found that consumer acceptance of plant protein-based meat alternatives is the highest, followed by cultured meat (Onwezen et al. (2021), Appetite, 159:105058). However, production of plant-based meat alternatives still faces some challenges such as the reconstruction of meat-like color, flavor, nutritional-value, and structure (Zhang et al. (2022), Curr. Opin. Food Sci. 43:43).

Genetic engineering represents an expedient strategy for upgrading plant-based recombinant protein products. These recombinant proteins in plants, when used as an ingredient, or used as a whole with the original plant part, can help substitute animal-based protein in the human diet and provide the desired organoleptic properties. Expression of recombinant proteins in plants can help upgrade its color, flavor, nutritional-value, and structure in its native form or when used as an ingredient. The present disclosure provides for a solution to produce critical animal heme proteins at a high expression level in transgenic plants and their seeds.

There have been some previous efforts that expressed plant-derived heme proteins in plants for further production of food products. For example, US patent application publication US2019292555 discloses rice and Arabidopsis transgenic plants expressing the soy leghemoglobin Lbc2 under the control of an alcohol inducible promoter. This published patent application does not show any data associated with the expression levels of the recombinant leghemoglobin.

The US patent application publication US2019292217 describes transgenic Arabidopsis thaliana plants overexpressing an enzyme involved in the heme biosynthetic pathway (glutamyl-tRNA reductase (GluTR) binding protein) as well as the expression of a soy leghemoglobin. The document does not provide any data about the expression levels of the leghemoglobin.

The international application publication WO2022072846 discloses information, but no experimentation, about transgenic plants expressing a heme protein with altered fatty acid profiles and upregulated heme biosynthesis.

The international application publication WO9902687 discloses a method to increase the content of iron in transgenic rice plants by expressing a rice- or an Arabidopsis thaliana-hemoglobin; however, the transgenic plants show a low hemoglobin expression level.

A study discloses the production of human myoglobin in leaves of Nicotiana benthamiana (Carlsson et al. (2020), Sci. Rep. 10:1). This document does not show data about heme loading to the recombinant myoglobin nor the functionality or correct structure fold of this recombinant protein nor its incorporation into food products.

However, these attempts to produce heme proteins in plants have not resulted in high levels (e.g., >5% tsp) of recombinant heme protein expression in plant seeds. Molecular farming studies, focused on expressing the gene via nuclear transformation, average expression levels of recombinant proteins of 0.5-2% tsp in stably transformed plants (Fischer & Emans, (2000), Transgenic Res. 9:279; Shanmugaraj et al. (2020), Plants, 9:842) These recombinant proteins produced in plants are mainly pharmaceutical proteins, proteins for diagnostic, research and cosmetic industries. In chloroplasts, researchers have achieved higher yields of recombinant protein expression, with ranges of 3-46% tsp from the plant (Dhingra & Daniell, (2006), Arabidopsis protocols, 245; Shanmugaraj et al, (2020), Plants, 9:842). The aforementioned recombinant proteins expressed in chloroplast are mainly pharmaceuticals but also include herbicide resistance genes. In seeds, recombinant proteins accumulate to a lower average concentration (0.05-1% tsp) (Jaeger et al, (2002). Nat. Biotechnol, 20:1265: Shanmugaraj et al. (2020), Plants, 9:842). However, independent studies have identified regulatory elements that produce significantly higher levels of protein in plant seeds (Jaeger et al. (2002), Nat. Biotechnol. 20:1265; Ishimoto et al. (2012), Biosci. Biotechnol. Biochem. 76:2142; Wadahama et al. (2012), Plant Physiol. 158:1395; Goossens et al. (1999), Plant Physiol. 120:1095; Diamos & Mason, (2018). Plant Biotechnol. J. 16:1971). These studies have reported up to 15-36% tsp in seeds of model species such as Arabidopsis and tobacco (Jaeger et al. (2002), Nat. Biotechnol. 20:1265; Goossens et al. (1999), Plant Physiol. 120:1095). However, none of these independent studies are focused on complex proteins such as the heme proteins in the present disclosure and are only validated in model plant species. Combinations of regulatory elements described herein have the potential to result in a stable seed protein production of more than 5%, 8%, or 10% tsp in commercially important seed crops such as legumes.

Therefore, state of the art still has not provided a solution to produce heme proteins at high expression levels in seeds of transgenic plants.

### BRIEF SUMMARY

In some aspects, provided herein is a transgenic plant, plant tissue, or plant cell comprising an exogenous nucleic acid encoding for a heme protein. In some aspects, said nucleic acid is operatively linked to a seed-specific promoter and a transcription terminator. In some aspects, said heme protein is expressed in a seed in an amount of at least about 5% total soluble protein (TSP).

In some aspects, said nucleic acid is operatively linked to a transcriptional or translational enhancer.

In some aspects, said heme protein is expressed in the seed in an amount of at least about 8% TSP.

In some aspects, said heme protein is expressed in the seed in an amount of at least about 10% TSP.

In some aspects, said heme protein comprises a plant derived heme protein, a microorganism derived heme protein, or an animal derived heme protein or a synthetic protein designed based on natural heme proteins.

In some aspects, said heme protein comprises heme proteins involved in oxygen transport, enzymes having a prosthetic heme group, or heme proteins involved in the electron transport chain.

In some aspects, said heme protein comprises hemoglobin, myoglobin, neuroglobin, cytoglobin, cytochrome P450s, cytochrome c oxidase, ligninases, catalase, peroxidases, cytochrome a, cytochrome b, or cytochrome c.

In some aspects, said heme protein is an animal derived heme protein selected from the group consisting of hemoglobin and myoglobin.

In some aspects, said seed specific promoter comprises the beta-conglycinin alpha subunit of the 7S storage (7s) promoter from soybean, the beta-phaseolin (Phas) promoter from common bean, USP promoter from Vicia faba, SBP promoter from Vicia faba, Legumin B4 promoter from Vicia faba, Napin promoter from Brassica napus, Vicilin promoter from Pisum sativum, α-globulin promoter from cotton, γ-zein promoter from maize, glutenin promoter from wheat. VvβVPE promoter from Vitis spp, Groundnut seed promoter (GSP) from peanut. 7αP promoter from soybean, AtLAC15 promoter from Arabidopsis thaliana, SSPs promoter from chickpea, Lectin promoter from soybean, Oleosin promoter from Brassica napus, AhLEC1A promoter from peanut. Glu-ID-1 promoter from wheat, Sesame 2S albumin (2Salb) promoter from sesame, or 8SGα promoter from mung bean.

In some aspects, the transgenic plant, plant tissue, or plant cell further comprises a terminator sequence. In some aspects, the terminator sequence comprises the Extensin terminator from tobacco, Ub10 terminator from Arabidopsis thaliana, Hsp70 terminator from Arabidopsis thaliana, Hsp18.2 terminator from Arabidopsis thaliana, Act2 terminator from Arabidopsis thaliana, G7 terminator from Arabidopsis thaliana, 3g24240 terminator from Arabidopsis thaliana, NOS terminator from Agrobacterium tumefaciens, Ocs terminator from Agrobacterium tumefaciens, Mas terminator from Agrobacterium tumefaciens, 35s terminator from Cauliflower Mosaic Virus, Rbc terminator from Chrysanthemum, Ags terminator from Agrobacterium tumefaciens, 3′ utr-nos terminator from Agrobacterium tumefaciens, 7s terminator from soybean. E9 terminator from Pisum sativum, ORF25 terminator from Agrobacterium tumefaciens, pinII terminator from Solanum tuberosum, tml terminator from Agrobacterium tumefaciens. Tr7 terminator from Agrobacterium tumefaciens, or the Arc5 terminator from Phaseolus vulgaris.

In some aspects, the transgenic plant, plant tissue, or plant cell further comprises a transcription or translation enhancer selected from the group consisting of: 5′ Untranslated Region (UTR) from Tobacco Etch Virus (TEV) and Rb7Mar 3′ Matrix Attachment Region as part of the transcription terminator.

In some aspects, the exogenous nucleic acid is operatively linked to a beta-conglycinin alpha subunit of the 7S storage protein (7s) promoter from soybean, and a NOS terminator.

In some aspects, the exogenous nucleic acid is operatively linked to a beta-conglycinin alpha subunit of the 7S storage protein (7S) promoter from soybean, and an Arc5 terminator and Rb7MAR fused to the Arc5 terminator.

In some aspects, the exogenous nucleic acid is operatively linked to a beta-conglycinin alpha subunit of the 7S storage protein (7S) promoter from soybean, a 5′ UTR TEV enhancer and an Arc5 terminator and Rb7MAR fused to arc5.

In some aspects, the exogenous nucleic acid is operatively linked to a beta-phaseolin (Phas) promoter from common bean, and a NOS terminator.

In some aspects, the exogenous nucleic acid is operatively linked to a beta-phaseolin (Phas) promoter from common bean, an Arc5 terminator fused with the Rb7MAR region.

In some aspects, the exogenous nucleic acid is operatively linked to a beta-phaseolin (Phas) promoter from common bean, a 5′ UTR TEV enhancer, and an Arc5 terminator fused with the Rb7MAR region.

In some aspects, said nucleic acid encoding for a heme protein comprises a nucleic acid sequence having at least 80% sequence identity to SEQ ID NO: 1. In some aspects, said nucleic acid encoding for a heme protein comprises a nucleic acid sequence having at least 80% sequence identity to SEQ ID NO: 2. In some aspects, said transgenic plant, plant tissue, or plant cell is derived from Glycine max. Oryza sativa, Hordeum vulgare. Zea mays, Secale cereale. Avena sativa, Beta vulgaris, Beta vulgaris subsp, vulgaris, Pastinaca sativa, Phaseolus vulgaris, Pisum sativum, Vigna angularis, Vigna radiata, Cicer arietinum, Arachis hypogaea, Lens culinaris, Medicago sativa. Eruca vesicaria, Brassica juncea. Lactuca sativa, Brassica, Solanum tuberosum, Ipomoea batatas, Manthot esculenta, Triticum aestivum or Triticum spelta.

In some aspects, provided herein is a method to obtain a recombinant heme protein. In some aspects, said method comprises i) providing a transgenic plant capable of expressing at least about 5% TSP of a heme protein in seeds; ii) cultivating said transgenic plant; iii) harvesting said transgenic plant; and iv) isolating and purifying the animal heme protein from said harvested plant.

In some aspects, the harvesting comprising harvesting the seeds of said transgenic plant.

In some aspects, provided herein is a transgenic seed comprising at least about 5% TSP of a recombinant heme protein.

In some aspects, provided herein is a transgenic seed comprising at least about 8% TSP of a recombinant heme protein.

In some aspects, provided herein is a transgenic seed comprising at least about 10% TSP of a recombinant heme protein.

In some aspects, said transgenic seed is from a species selected from the group consisting of Glycine max, Oryza sativa, Hordeum vulgare. Zea mays, Secale cereale, Avena sativa, Beta vulgaris. Beta vulgaris subsp. vulgaris, Pastinaca sativa, Phaseolus vulgaris, Pisum sativum, Vigna angularis. Vigna radiata, Cicer arietinum. Arachis hypogaea, Lens culinaris, Medicago sativa, Eruca vesicaria, Brassica juncea, Lactuca sativa, Brassica, Solanum tuberosum, Ipomoea batatas, Manihot esculenta, Triticum aestivum and Triticum spelta.

In some aspects, said recombinant heme protein is an animal heme protein.

In some aspects, said recombinant heme protein is myoglobin.

In some aspects, said recombinant heme protein is hemoglobin.

In some aspects, provided herein is a food composition comprising any of the transgenic seeds disclosed herein.

In some aspects, provided herein is a food composition comprising the heme protein of any of the plants, plant tissues, or plant cells disclosed herein.

In some aspects, provided here is a meat analogue food composition comprising any of the transgenic seeds disclosed herein.

In some aspects, provided here is a meat analogue food composition comprising the heme protein of any of the plants, plant tissues, or plant cells disclosed herein.

In some aspects, the present disclosure also provides a polynucleotide comprising a nucleic acid encoding for a heme protein, wherein said nucleic acid is operatively linked to a seed-specific promoter selected from the group consisting of beta-conglycinin alpha subunit of the 7S storage (7s) promoter from soybean, the beta-phaseolin (Phas) promoter from common bean, USP promoter from Vicia faba. SBP promoter from Vicia faba. Legumin B4 promoter from Vicia faba, Napin promoter from Brassica napus, Vicilin promoter from Pisum sativum, α-globulin promoter from cotton, γ-zein promoter from maize, glutenin promoter from wheat, VvβVPE promoter from Vitis spp. Groundnut seed promoter (GSP) from peanut, 7αP promoter from soybean, AtLAC15 promoter from Arabidopsis thaliana, SSPs promoter from chickpea. Lectin promoter from soybean. Oleosin promoter from Brassica napus. AhLEC1A promoter from peanut, Glu-ID-1 promoter from wheat, Sesame 2S albumin (2Salb) promoter from sesame, and 8SGα promoter from mung bean.

In some aspects, said heme protein comprises a plant derived heme protein, a microorganism derived heme protein, or an animal derived heme protein.

In some aspects, said nucleic acid encoding for a heme protein comprises a nucleic acid sequence having at least 80% sequence identity to SEQ ID NO: 1 or SEQ NO: 2.

In some aspects, the polynucleotide comprises a nucleic acid sequence having at least 80% sequence identity to any one of SEQ ID NOs: 3 to 7.

In some aspects, said nucleic acid further comprises a transcription terminator selected from the group consisting of: Extensin terminator from tobacco, Ub10 terminator from Arabidopsis thaliana, Hsp70 terminator from Arabidopsis thaliana, Hsp18.2 terminator from Arabidopsis thaliana, Act2 terminator from Arabidopsis thaliana, G7 terminator from Arabidopsis thaliana, 3g24240 terminator from Arabidopsis thaliana. NOS terminator from Agrobacterium tumefaciens, Ocs terminator from Agrobacterium tumefaciens, Mas terminator from Agrobacterium tumefaciens, 35s terminator from Cauliflower Mosaic Virus, Rbc terminator from Chrysanthemum, Ags terminator from Agrobacterium tumefaciens. 3′ utr-nos terminator from Agrobacterium tumefaciens. 7s terminator from soybean, E9 terminator from Pisum sativum, ORF25 terminator from Agrobacterium tumefaciens, pinII terminator from Solanum tuberosum, tml terminator from Agrobacterium tumefaciens, Tr7 terminator from Agrobacterium tumefaciens, and the Arc5 terminator from Phaseolus vulgaris.

In some aspects, said nucleic acid further comprises a transcriptional or translational enhancer selected from the group consisting of 5′ UTR TEV and Rb7Mar 3′ Matrix Attachment Region.

In some aspects, the present disclosure is directed to an expression vector comprising a nucleic acid encoding for a heme protein, wherein said nucleic acid is operatively linked to a seed-specific promoter selected from the group consisting of beta-conglycinin alpha subunit of the 7S storage (7s) promoter from soybean, the beta-phaseolin (Phas) promoter from common bean. USP promoter from Vicia faba, SBP promoter from Vicia faba. Legumin B4 promoter from Vicia faba, Napin promoter from Brassica napus, Vicilin promoter from Pisum sativum, α-globulin promoter from cotton, Y-zein promoter from maize, glutenin promoter from wheat, VvβVPE promoter from Vitis spp, Groundnut seed promoter (GSP) from peanut, 7αP promoter from soybean, AtLAC15 promoter from Arabidopsis thaliana, SSPs promoter from chickpea, Lectin promoter from soybean. Oleosin promoter from Brassica napus, AhLEC1A promoter from peanut, Glu-ID-1 promoter from wheat, Sesame 2S albumin (2Salb) promoter from sesame, or 8SGα promoter from mung bean.

In some aspects, the expression vector comprises a heme protein derived from a microorganism, a plant or an animal.

In some aspects, the expression vector comprises a nucleic acid coding for heme protein with a sequence having at least 80% sequence identity to SEQ ID NO: 1.

In some aspects, the expression vector comprises a nucleic acid coding for heme protein with a sequence having at least 80% sequence identity to SEQ ID NO: 2.

In some aspects, the expression vector comprises a nucleic acid coding for heme protein with a sequence having at least 80% sequence identity to SEQ ID NO: 1 operatively linked to a beta-phaseolin (Phas) promoter.

In some aspects, the expression vector comprises a nucleic acid coding for heme protein with a sequence having at least 80% sequence identity to SEQ ID NO: 2 operatively linked to a beta-phaseolin (Phas) promoter. In some aspects, the expression vector, further comprises a transcription terminator selected from the group consisting of: Extensin terminator from tobacco. Ub10 terminator from Arabidopsis thaliana, Hsp70 terminator from Arabidopsis thaliana, Hsp18.2 terminator from Arabidopsis thaliana, Act2 terminator from Arabidopsis thaliana, G7 terminator from Arabidopsis thaliana, 3g24240 terminator from Arabidopsis thaliana, NOS terminator from Agrobacterium tumefaciens, Ocs terminator from Agrobacterium tumefaciens, Mas terminator from Agrobacterium tumefaciens. 35s terminator from Cauliflower Mosaic Virus, Rbc terminator from Chrysanthemum, Ags terminator from Agrobacterium tumefaciens, 3′ utr-nos terminator from Agrobacterium tumefaciens, 7s terminator from soybean, E9 terminator from Pisum sativum, ORF25 terminator from Agrobacterium tumefaciens, pinII terminator from Solanum tuberosum, tml terminator from Agrobacterium tumefaciens, Tr7 terminator from Agrobacterium tumefaciens, and the Arc5 terminator from Phaseolus vulgaris.

In some aspects, the expression vector, comprises a nucleic acid coding for heme protein with a sequence having at least 80% sequence identity to SEQ ID NO: 1 operatively linked to a beta-phaseolin (Phas) promoter and Arc5 terminator.

In some aspects, the expression vector, comprises a nucleic acid coding for heme protein with a sequence having at least 80% sequence identity to SEQ ID NO: 2 operatively linked to a beta-phaseolin (Phas) promoter and Arc5 terminator.

In some aspects, the expression vector, further comprises a transcriptional or translational enhancer selected from the group consisting of 5′ UTR TEV and Rb7Mar 3′ Matrix Attachment Region.

In some aspects, the expression vector, comprises a nucleic acid coding for heme protein with a sequence having at least 80% sequence identity to SEQ ID NO: 1 operatively linked to a beta-phaseolin (Phas) promoter, an Arc5 terminator and a Rb7Mar 3′ Matrix Attachment Region.

In some aspects, the expression vector, comprises a nucleic acid coding for heme protein with a sequence having at least 80% sequence identity to SEQ ID NO: 2 operatively linked to a beta-phaseolin (Phas) promoter, an Arc5 terminator and a Rb7Mar 3′ Matrix Attachment Region.

In some aspects, the expression vector comprises a nucleic acid sequence having at least 80% sequence identity to any one of SEQ ID NOs: 3 to 7.

### BRIEF DESCRIPTION OF THE DRAWINGS

FIG. 1 (FIG. 1) illustrates pIPTRA0: p35S+HbA-LL-HbB binary vector. This plasmid allows the expression of the hemoglobin (HbA-LL-HbB) gene driven by constitutive promoter CaMV35S, and includes the Nopaline synthase terminator (TNOS). The hemoglobin gene consists of the alpha-globin and beta-globin linked via a long linker (LL) of 63 bp. The vector backbone region is 11,238 bp long. The HbA-LL-HbB consists of a soybean codon-optimized sequence.

FIG. 2 (FIG. 2) depicts pIPTRA0: p7S+HbA-LL-HbB-Arc5T binary vector. This plasmid allows the expression of the hemoglobin (HbA-LL-HbB) gene driven by 7S globulin (7s) promoter, in conjunction with the ARC5 terminator and Rb7 matrix array region (MAR). The hemoglobin gene consists of the alpha-globin and beta-globin linked via a long linker (LL) of 63 bp. The vector backbone region is 12,159 bp long. The HbA-LL-HbB consists of a soybean codon-optimized sequence.

FIG. 3 (FIG. 3) shows the 35S+TEV+myoglobincDNA+NOS linear construct, referred to as EC1. This linear construct allows the expression of the myoglobin gene driven by constitutive promoter CaMV35S promoter, and includes the Nopaline synthase terminator (TNOS). The linear construct is 1.655 bp long. The myoglobin gene consists of a soybean codon-optimized sequence.

FIG. 4 (FIG. 4) illustrates the p7S+TEV+myoglobincDNA+arc5+Rb7MAR linear construct, referred to as EC2. This linear construct allows the expression of the myoglobin gene driven by the 7S globulin promoter, in conjunction with the ARC5 terminator and Rb7 matrix array region (MAR). The linear construct is 3,220 bp long. The myoglobin gene consists of a soybean codon-optimized sequence.

FIG. 5 (FIG. 5) depicts the PPhas+myoglobincDNA+arc5+Rb7MAR linear construct, referred to as EC3. This linear construct allows the expression of the myoglobin gene driven by the phas promoter, in conjunction with the ARC5 terminator and Rb7 matrix array region (MAR). Phas promoter includes the seed specific enhancer (SSE). The linear construct is 2.623 bp long. The myoglobin gene consists of a soybean codon-optimized sequence.

FIG. 6 (FIG. 6) depicts T-DNA nucleotide sequence of pIPTRA0: p35S+HbA-LL-HbB (5′ to 3′) (4728 bp). Tvsp transcription terminator (italics) Bar gene cDNA, conferring resistance to glufosinate-ammonium, complementary orientation (underlined). Tobacco Etch Virus (TEV) Translational Enhancer, complementary orientation (bolded). CaMV35S-derived double Promoter pr2×35S, complementary orientation (shaded italics). Tnos Transcriptional terminator (shaded underlined). HbA-LL-HbB CDS, complementary orientation (shaded and bolded). Hemoglobin gene consists of the alpha-globin and beta-globin linked via a long linker (LL) (black background with white text) of 63 bp.

FIG. 7 (FIG. 7) depicts T-DNA nucleotide sequence of pIPTRA0: p7S+HbA-LL-HbB-Arc5T (5′ to 3′) (5649 bp). Tvsp transcription terminator (Italics). Bar gene cDNA, conferring resistance to glufosinate-ammonium, complementary orientation (underlined). Tobacco Etch Virus (TEV) Translational Enhancer, complementary orientation (bolded). CaMV35S-derived double Promoter pr2×35S, complementary orientation (shaded italics). Tobacco Rb7 Matrix Attachment Region, complementary orientation (shaded underline). Arcelin-5 Transcriptional Terminator, complementary orientation (bolded and shaded). HbA-LL-HbB CDS, complementary orientation (black background, white text, italics). Hemoglobin gene consists of the alpha-globin and beta-globin linked via a long linker (LL) (lower case and black background with white text) of 63 bp. Tobacco Etch Virus (TEV) Translational Enhancer, complementary orientation. 7S Globulin Promoter, complementary orientation (black background with white text, underlined).

FIG. 8 (FIG. 8) shows the nucleotide sequence of EC1 linear construct (5′ to 3′) (1,655 bp). CaMV35S-derived double Promoter 2×35S (italics). Tobacco Etch Virus (TEV) Translational Enhancer (underlined). Myoglobin CDS (bolded). TNOS Transcriptional terminator (shaded italics).

FIG. 9 (FIG. 9) shows the nucleotide sequence of EC2 linear construct (5′ to 3′) (3.220 bp). 7S globulin (7s) promoter (italics). Tobacco Etch Virus (TEV) Translational Enhancer (underlined). Myoglobin CDS (bolded). ARC5 terminator and Rb7 matrix array region (shaded italics).

FIG. 10 (FIG. 10) shows the nucleotide sequence of EC3 linear construct (5′ to 3′) (2,623 bp). Phas promoter (italics). Myoglobin CDS (underlined). ARC5 terminator and Rb7 matrix array region (bolded).

FIGS. 11A-11B (FIGS. 11A-11B) depict the regeneration (11A, 11B) of soybean transgenic lines. Image A and B show shoots regenerating on selection media transformed with pIPTRA0: p7S+HbA-LL-HbB-Arc5T and EC3, respectively. Shoots from Image A and B were transformed via agrobacterium and biolistic transformation methods, respectively.

FIG. 12 (FIG. 12) shows PCR amplification results from events transformed with pIPTRA0: p35S+HbA-LL-HbB binary vector. Specific primers were designed for the detection of the 2×35S promoter and HbA CDS. The expected amplicon length is 412 bp. MM: molecular marker (100 bp). 463 to 472 samples are a batch of potential transgenic plants produced in study. C−1 and C−2 are negative controls prepared during DNA extraction. C+1 to C+2 are positive controls for pIPTRA0: p35S+HbA-LL-HbB binary vector from two plant samples characterized previously. C+3 was amplified from the pIPTRA0: p35S+HbA-LL-HbB binary vector. C−3 DNA represents another binary vector (negative control). Bco: blank of PCR. WT: wild type soybean DNA

FIG. 13 (FIG. 13) shows PCR amplification results from events transformed with pIPTRA0: p7S+HbA-LL-HbB-Arc5T binary vector. Specific primers were designed for the detection of the 7S promoter and HbA CDS. The expected amplicon length is 390 bp. MM: molecular marker (100 bp). 563 to 562 samples are a batch of potential transgenic plants produced in study. C−1 is a negative control prepared during DNA extraction. Bco is a blank for PCR. WT: wild type soybean DNA. C+1 is a positive sample for pIPTRA0: p7S+HbA-LL-HbB-Arc5T that had been characterized previously. C−3 DNA represents another binary vector (negative control). C+2 was amplified from the pIPTRA0: p7S+HbA-LL-HbB-Arc5T binary vector.

FIG. 14 (FIG. 14) shows qPCR amplification results from events transformed with EC1, EC2, and EC3 vectors. Specific primers were designed for the detection of the aadA1a CDS. Red line: Marker Ct cutoff, positives must be <25 Ct value. WT Ctrl: wild type soybean DNA. Water Ctrl: water control. C+1 and C+2 are positive controls for the aadA1 that had been characterized previously. Samples 1 to 4 for EC1, EC2, and EC3 are batches of potential transgenic plants produced in study.

FIGS. 15A-15C (FIGS. 15A-15C) depict protein extracts coloration from soybean seeds of WT (FIG. 15A), pIPTRA0: p35S+HbA-LL-HbB (FIG. 15B), and pIPTRA0: p7S+HbA-LL-HbB-Arc5T (FIG. 15C) transgenic events. Protein extractions from FIG. 15C show a pink coloration (darker shading in the black and white image of FIG. 15C) likely attributed to the expression and presence of the heterologous hemoglobin gene. An arrow and circles were added to point at the darker shading.

FIGS. 16A-16C (FIGS. 16A-16C) show coloration of soybean half-cut seeds (FIG. 16A) and of protein extracts from soybean seeds (FIG. 16B, 16C). Half-cut seeds are shown for WT seeds (left side of panel FIG. 16A) and for a transgenic EC3 event (right side of panel FIG. 16A). Protein seed extractions are shown for WT seeds (FIG. 16B) and for transgenic EC3 events (FIG. 16C). Pink coloration shown in FIGS. 16A and 16C, (darker shading in the black and white images of FIGS. 16A and 16C) corresponding to EC3 transgenic lines, is likely attributed to the expression and presence of the heterologous porcine myoglobin gene.

FIGS. 17A-17C (FIGS. 17A-17C) illustrate three replicates of Western blot (17A, 17B, 17C) analyses of soybean seeds. Soybean seed protein extracts were run on 12% SDS PAGE gels. Protein bands were then transferred to a nitrocellulose membrane and western blots were developed with the anti-porcine antibody. White dots indicate control hemoglobin bands as well as putative hemoglobin bands at 16 kDa (monomer), 32 kDa (dimer), and 64 kDa (tetramer). Lane 1:100 μg of WT extract. Lanes 2 to 6: WT extract+10, 25, 50, 150, and 250 ng of Hb standard, respectively. Lanes 7 to 14:100 μg protein extract of EC3 events 63, 64, 65, 66, 67, 68, 69, 70. Lane 15: Molecular weight standard.

FIG. 18 (FIG. 18) depicts hemoglobin expression levels in seeds of soybean transgenic events. Total soluble protein (% TSP), represented in the Y axis, was obtained via Western blot quantitative analysis. Expression of events transformed with pIPTRA0: p35S+HbA-LL-HbB binary vector (events 15 and 19) are shown in black bars. Expression of events transformed with pIPTRA0: p7S+HbA-LL-HbB-Arc5T binary vector (events 46 to 75) are shown in white bars. WT and empty vector events are not shown in the graph because the expression values did not reach the limit of detection (LOD). The extracts were run on 12% SDS PAGE gels, then transferred to a nitrocellulose membrane and western blots were developed with the anti-porcine antibody. The percentage hemoglobin of TSP was quantified by comparing the intensity of the hemoglobin bands from the seed extracts with that from the hemoglobin standards. Each event consisted of one biological replicate (a pool of 3 seeds) with 3 technical replicates. Standard deviation is presented as error bars for each event.

FIG. 19 (FIG. 19) shows the results from porcine hemoglobin identification and quantification from whole soybean seed protein extracts via Liquid chromatography-mass spectrometry (LC-MS) Normalized pig hemoglobin levels are shown in the graph for WT (Wild type) as well as events 54 and 63 (both transformed with pIPTRA0: p7S+HbA-LL-HbB-Arc5T). No hemoglobin was detected in the WT sample. No replicates were run.

FIGS. 20A-20B (FIGS. 20A-20B) show myoglobin expression levels in soybean transgenic events. Total soluble protein (% TSP), represented in the Y axis, was obtained via ELISA quantitative analysis. FIG. 20A shows expression values for WT, and events transformed with empty vector (1542 event series). EC1 (1545 event series), and EC2 (1544 event series). FIG. 20B shows expression values for events transformed with EC3 (1543 event series) as well as WT and events transformed with empty vector. EC1 and EC2. Myoglobin quantitation from seed extracts was done using the Alpha Diagnostics ELISA kit (cat #600-640-PMY). All samples were normalized to 50 μg/mL total soluble protein (TSP) and tested for myoglobin content according to the manufacturer's protocols. The concentration of myoglobin was determined by reference to the standard curve. Each event consisted of one biological replicate (a pool of 3 seeds) with 3 technical replicates. Standard deviation is presented as error bars for each event.

### DETAILED DESCRIPTION

The terminology used herein is for the purpose of describing particular aspects only and is not intended to be limiting.

It is to be noted that the term “a” or “an” entity refers to one or more of that entity: for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B.” “A or B,” “A” (alone), and “B” (alone). Likewise, the term “and/or” as used in a phrase such as “A, B, and/or C” is intended to encompass each of the following aspects: A, B, and C; A, B, or C: A or C: A or B: B or C: A and C; A and B: B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

Throughout this disclosure, various aspects of this disclosure are presented in a range format. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

“Percent identity” refers to the extent of identity between two sequences (e.g., amino acid sequences or nucleic acid sequences). Percent identity can be determined by aligning two sequences, introducing gaps to maximize identity between the sequences. Alignments can be generated using programs known in the art. For purposes herein, alignment of nucleotide sequences can be performed with the blastn program set at default parameters, and alignment of amino acid sequences can be performed with the blastp program set at default parameters (see National Center for Biotechnology Information (NCBI) on the worldwide web, ncbi.nlm.nih.gov).

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In some aspects, the production of heme proteins in transgenic plants as well as the use of these heme proteins for alternative meats are discussed herein. In the following description, for purposes of explanation, numerous specific details are set forth in order to provide a thorough understanding of the present invention. It will be evident, however, to one skilled in the art that the present invention may be practiced without these specific details.

The term “derived from,” as used herein, refers to a component that is isolated from or made using a specified molecule or organism, or information (e.g., amino acid or nucleic acid sequence) from the specified molecule or organism. For example, a nucleic acid sequence (e.g., an expression vector) that is derived from a second nucleic acid sequence can include a nucleotide sequence that is identical or substantially similar to the nucleotide sequence of the second nucleic acid sequence.

“Nucleic acid,” “polynucleotide,” and “oligonucleotide,” are used interchangeably in the present application. These terms refer only to the primary structure of the molecule. Thus, these terms include double- and single-stranded DNA, as well as double- and single-stranded RNA. The terms “nucleic acid.” “polynucleotide.” and “oligonucleotide.” as used herein, are defined as it is generally understood by the skilled person as a molecule comprising two or more covalently linked nucleotides. Such covalently bound nucleotides can also be referred to as nucleic acid molecules or oligomers.

Polynucleotides can be made recombinantly, enzymatically, or synthetically. e.g., by solid-phase chemical synthesis followed by purification. When referring to a sequence of the polynucleotide or nucleic acid, reference is made to the sequence or order of nucleobase moieties, or modifications thereof, of the covalently linked nucleotides.

As used herein, the term “heme proteins” include proteins that have the ability of binding a heme prosthetic group to their structure. As used herein, the term heme protein also refers to critical components of flesh of an animal and/or animal proteins, and can provide color and taste to plant-based meat products. Myoglobin and hemoglobin, considered heme proteins, are oxygen-binding proteins in animals. Also, as used herein, the term heme protein refers to heme containing proteins, wherein the term containing means the protein is linked through covalent or non-covalent bonds to the protein. As used herein, the term heme protein refers to not only the full-length protein but also fragments or variants thereof.

As used herein the term “animal heme protein” or “animal derived heme protein” refers to heme proteins expressed in animals, but excludes the human derived heme proteins. According to some aspects of the present disclosure, the animal heme proteins comprise heme proteins involved in the oxygen transport, such as hemoglobin, myoglobin, neuroglobin, and cytoglobin; enzymes having a prosthetic heme group, such as cytochrome P450s, cytochrome c oxidase, ligninases, catalase, and peroxidases, as well as heme proteins involved in the electron transport chain, such as cytochrome a, cytochrome b, and cytochrome c.

As used herein, the term “plant derived heme protein”, means heme proteins whose genetic source is native from monocot or dicot plants such as Nicotiana tabacum or Nicotiana sylvestris (tobacco); Zea mays (corn), Arabidopsis thaliana, a legume such as Glycine max (soybean), Cicer arietinum (garbanzo or chickpea), Pisum sativum (pea), Phaseolus vulgaris (common bean) Vigna unguiculata (cowpea). Vigna radiata (mung beans), Lupinus albus (lupin), or Medicago sativa (alfalfa), Brassica napus (canola); Triticum sps. (wheat, including wheat berries, and spelt); Gossypium hirsutum (cotton); Oryza sativa (rice); Zizania sps. (wild rice); Helianthus annuus (sunflower); Beta vulgaris (sugarbeet); Pennisetum glaucum (pearl millet); Chenopodium sp. (quinoa); Sesamum sp. (sesame); Linum usitatissimum (flax); or Hordeum vulgare (barley).

As used herein, the term “microorganism derived heme protein”, means heme proteins whose genetic source is native from bacteria yeast, fungi such as Escherichia coli, Bacillus subtilis, Bacillus licheniformis. Bacillus megaterium, Synechocystis sp., Aquifex aeolicus, Methylacidiphilum infernorum. Thermophilus spp, A. eutrophus, Saccharomyces cerevisiae, Vitreoscilla sp. Pichia pastoris. Magnaporthe oryzae. Fusarium graminearum, Aspergillus oryzae, Trichoderma reesei. Myceliopthera thermophile, Kluyveromyces lactis, and Fusarium oxysporum.

As used herein, the term “recombinant protein” refers to a protein encoded by a gene (e.g., recombinant DNA) that has been cloned in a system that supports expression of the gene and translation of messenger RNA. Recombinant proteins are foreign proteins produced in expression hosts. Modification of the gene by recombinant DNA technology can lead to expression of a mutant protein.

As used herein, the term “recombinant heme protein” refers to a recombinant protein, where the recombinant protein is codified by foreign cDNA encoding for the heme protein. As used herein, the term “exogenous nucleic acid” means a cDNA coding for the recombinant heme protein; also, the term “exogenous nucleic acid” is used herein interchangeably with “recombinant nucleic acid”. The sequences and structure of numerous heme-containing polypeptides are known (Reedy, et al. (2007), Nucleic Acids Res. 6: D307).

The term “plant” includes reference to whole plants, plant organs, plant tissues, and plant cells, and progeny of the same and includes all monocots and dicots. The word plant used herein, also includes seeds, plant progeny, propagules whether sexually or asexually, descendants of these, such as cuttings or seed, as well as pre-harvest and post-harvest tissues and organs.

The term “transgenic plant” or “genetically engineered” means a plant that has been transformed with one or more exogenous nucleic acids (recombinant sequences). The term “transformation” refers to a process by which a recombinant sequence is introduced and expressed in a plant cell. In plant stable transformation, the foreign DNA is fully integrated into the host genome and remains integrated and continues to be expressed in later generations of the plant. In plant transient transformation, the foreign DNA is not integrated into the host genome and it is not expressed in later generations of the plant. Transformation may occur through Agrobacterium-inoculation, viral infection, electroporation, heat shock, lipofection, polyethylene glycol treatment, microinjection, silica beads, carbon nanotubes and particle bombardment methods.

In some aspects, the transgenic plant is a soy (Glycine max) plant. In some aspects, the genetically engineered plant is selected from the group consisting of: rice (Oryza sativa), barley (Hordeum vulgare), wheat (Triticum aestivum), corn (Zea mays), rye (Secale cereale), oat (Avena sativa), beet (Beta vulgaris), sugar beet (Beta vulgaris subsp. vulgaris), parsnip (Pastinaca sativa), bean, leafy vegetable, tuber, and grass. In some aspect, the bean is bean or pinto bean (Phaseolus vulgaris), pea (Pisum sativum), adzuki (Vigna angularis), mung (Vigna radiata), chickpea (Cicer arietinum), peanut (Arachis hypogaea), or lentil (Lens culinaris). In some aspects, the leafy vegetable is alfalfa (Medicago sativa), arugula (Eruca vesicaria), mustard (Brassica juncea), lettuce (Lactuca sativa), or Brassica. In some aspects, the tuber is a potato (Solanum tuberosum), a sweet potato (Ipomoea batatas), or a cassava (Manihot esculenta). In some aspects, the grass is triticale (Triticum aestivum) or spelt (Triticum spelta).

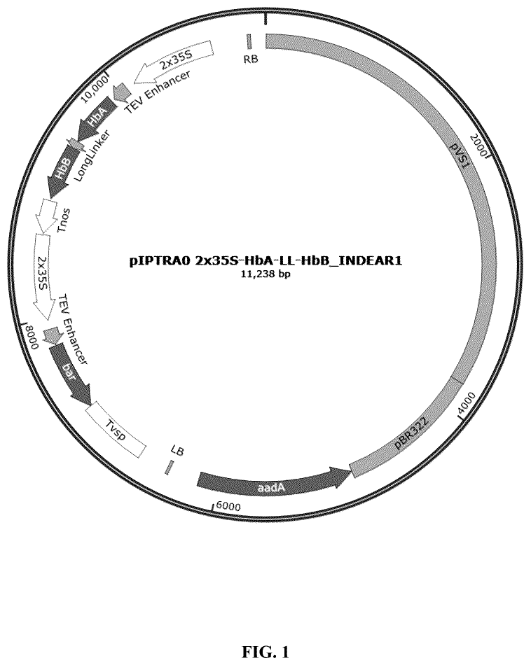

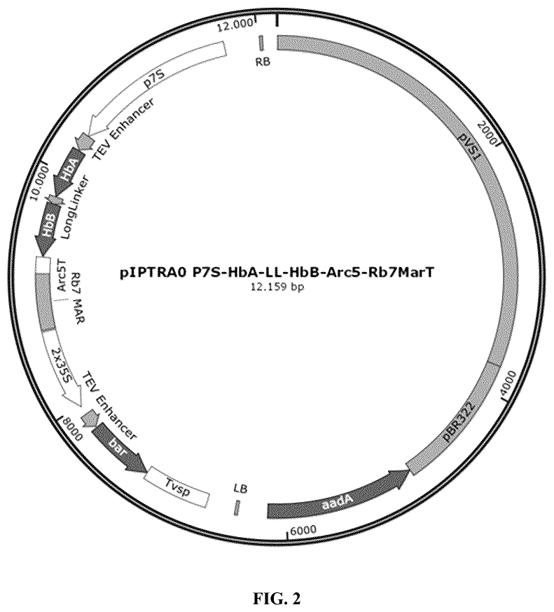

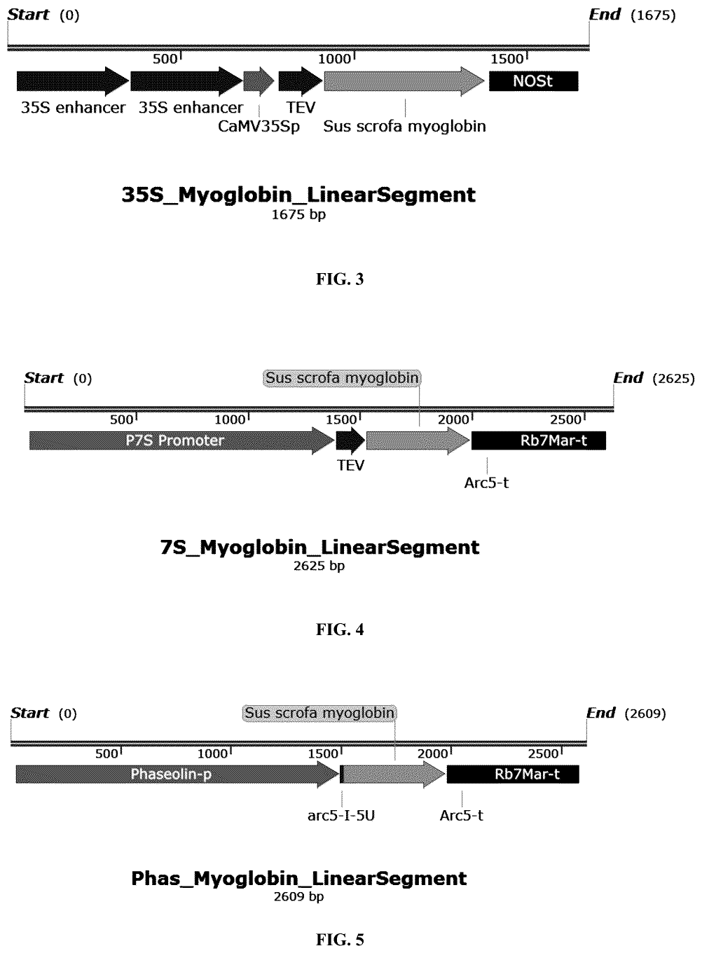

Illustrative recombinant sequences of the disclosure are provided in FIGS. 1-10. These recombinant sequences are contained within a plant transformation vector. In some aspects, these vectors are introduced into the Agrobacterium tumefaciens as circular plasmids. The t-DNA insert from the circular plasmids is introduced into plant cells via Agrobacterium-mediated transformation (FIGS. 1-2, 6-7, and SEQ ID NOs. 3-4). In some aspects, these vectors are bombarded into the plant cells as linear constructs (FIGS. 3-5, 8-10, and SEQ ID NOs. 5-7). In some aspects, a recombinant sequence comprises a promoter, an enhancer sequence, a sequence encoding for a heme protein, and a terminator (FIGS. 1, 3, 6, and 8). In some aspects, a recombinant sequence comprises a promoter, an enhancer sequence, a sequence encoding for a heme protein, a terminator, and a matrix attachment region (FIGS. 2, 4, 5, 7, 9, and 10). In some aspects, the heme protein is hemoglobin (FIGS. 1-2, and 6-7). In other aspects, the heme protein is myoglobin (FIGS. 3-5, and 8-10).

In some aspects, the recombinant sequence comprises a sequence named promoter that refers to nucleic acid sequences that promotes initiation of transcription. The promoter may be a constitutive promoter. A constitutive promoter is capable of initiating transcription in plant cells under any circumstances and its activity is not affected by environmental conditions. Some promoters are tissue specific because these promoters preferentially initiate transcription in certain organs. Other promoters are inducible, modulated by external stimuli such as different chemical, biotic and abiotic environmental factors.

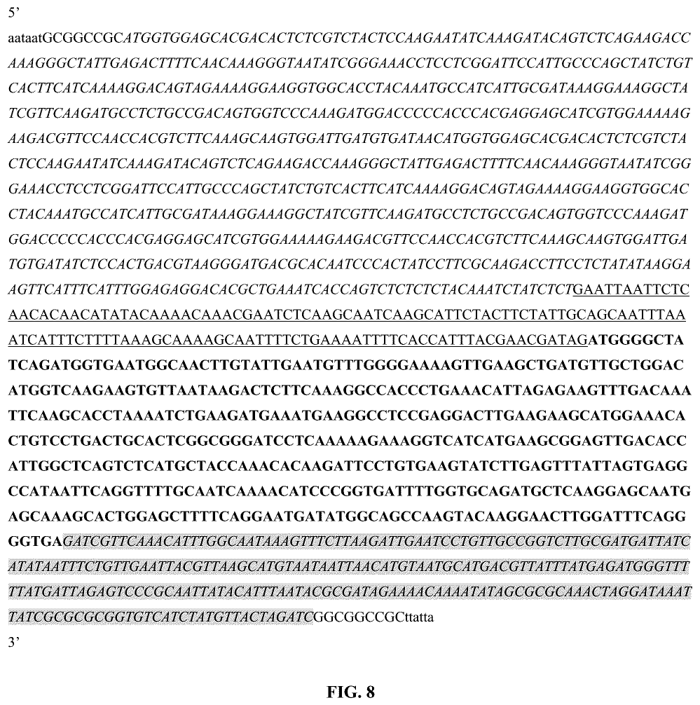

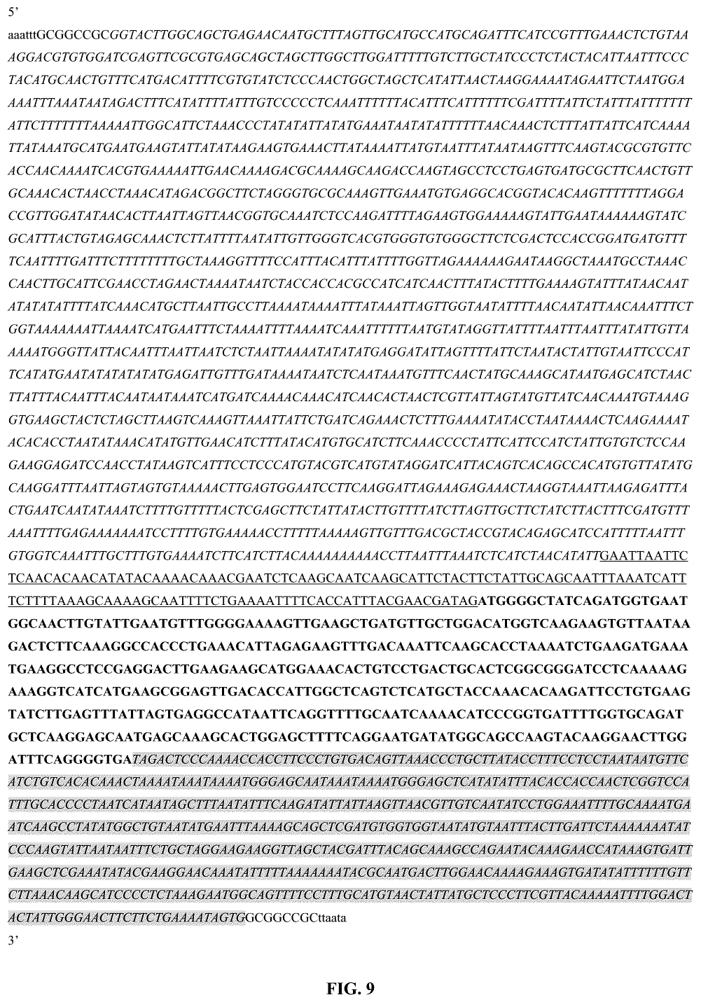

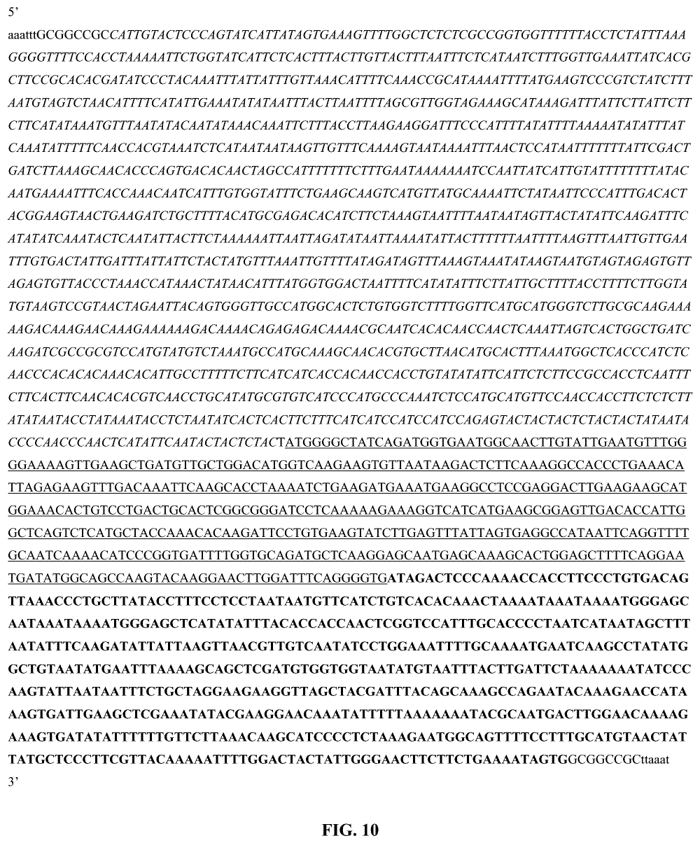

In some aspects, the promoter is a constitutive promoter such as the 35S promoter present as a double unit in tandem (2×35S promoter) (SEQ ID NO: 8) (FIGS. 1, 3, 6, and 8) derived from the cauliflower mosaic virus (CaMV). In some aspects, the promoter is a tissue specific promoter. The tissue specific promoter could be the beta-conglycinin alpha′ subunit of the 7S storage protein (7s) promoter from soybean (Zakharov et al. (2004). J. Exp. Bot. 55:1463) (SEQ ID NO: 11), beta-phaseolin (phas) promoter from common bean (Zakharov et al. (2004), J. Exp. Bot. 55:1463), USP promoter from Vicia faba (Zakharov et al. (2004), J. Exp. Bot. 55:1463), SBP promoter from Vicia faba (Zakharov et al. (2004), J. Exp. Bot. 55:1463), Legumin B4 promoter from Vicia faba (Zakharov et al. (2004), J. Exp. Bot. 55:1463), Napin promoter from Brassica napus (Vigeolas et al. (2007). Plant Biotechnol. J. 5:431), Vicilin promoter from Pisum sativum (Arun et al. (2014). Appl. Biochem. Biotechnol. 172:1763), α-globulin promoter from cotton (Sunilkumar et al. (2002), Transgenic Res. 11:347), γ-zein promoter from maize (Marzábal et al. (1998). Plant J. 16:41). Glutenin promoter from wheat (Lamacchia et al (2001), J. Exp. Bot. 52:243). VvβVPE promoter from Vitis spp (Gong et al. (2019). Planta, 250:657), Groundnut seed promoter (GSP) from peanut (Sunkara et al. (2014). Appl Biochem Biotechnol. 172:325), 7αP promoter from soybean (Fu et al. (2009); Northwest Sci. 37:105). AtLAC15 promoter from Arabidopsis thaliana (El-Mezawy et al. (2009). Biotechnol. Lett. 31:1961), SSPs promoter from chickpea (Verma & Bhatia, (2019), Funct. Integr. Genomics, 19:373), Lectin promoter from soybean (Ma et al. (2008), J. Plant Growth Regul. 27:68). Oleosin promoter from Brassica napus (Keddie et al. (1994) Plant Mol. Biol. 24:327), AhLEC1A promoter from peanut (Tang et al. (2021) PloS one. 16: e0242949), Glu-ID-1 promoter from wheat (Lamacchia et al. (2001). J. Exp. Bot. 52:243), Sesame 2S albumin (2Salb) promoter from sesame (Bhunia et al. (2014), Plant Mol. Biol. 86:351). 8SGα promoter from mung bean (Chen et al. (2014), J. Biotechnol. 174:49). In some aspects, the seed specific promoters are 7S (FIGS. 2, 4, 7, and 9) (SEQ ID NO: 11) and phas (FIGS. 5, and 10). Constructs based on either the 7s or the phas promoters show higher expression of protein in seeds.

The 7s and beta-phaseolin proteins are highly expressed seed storage proteins and their expression patterns have been characterized (Chandrasekharan et al. (2003), Plant J. 33:853; Hayashi et al. (2009). J. Hered. 100:802). The 7S Globulin gene (β-conglycinin) is a major seed-storage protein in soybean (Glycine max) This gene consists of three subunits: alpha, alpha′, and beta and comprises 30-35% of the total seed protein (Thanh and Shibasaki, (1976), Biochim. Biophys. Acta. 439:326; Hayashi et al. (2009), J. Hered. 100:802). The 7S promoter was inserted in soybean to express a human growth factor and the transgenic lines yielded 2.3% tsp (total soluble protein) for the recombinant protein. 38× higher than the 35S promoter (Ding et al. (2006), Biotechnol. Lett. 28:869). A human bone morphogenetic protein was expressed under the control of the 7s promoter resulting in yields of up to 9.28% tsp (Queiroz et al., 2019, Plant Mol. Biol. 96:429).

The phas gene encodes the major seed storage protein in Phaseolus vulgaris. Studies have found that the phas gene is highly expressed in the cotyledons during embryogenesis (Li et al. (1999), PNAS, 95:4772; Chandrasekharan et al. (2003), Plant J. 33:853). This gene is stringently turned off during all vegetative stages of plant development (Li et al. (1999), PNAS, 95:4772). In arabidopsis seeds and under the control of the phas promoter, expression levels of the recombinant protein reached up to 36% of total soluble seed protein (Jaeger et al. (2002), Nat. Biotechnol. 20:1265).

In some aspects, the expression cassette comprises a 2×35S promoter. In some aspects, the 2×35S promoter comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 8.

In some aspects, the 2×35S promoter comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 8.

In some aspects, the expression cassette comprises a 7S promoter. In some aspects, the 7S promoter comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 11.

In some aspects, the 7S promoter comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 11.

In some aspects, the expression cassette comprises a Phas promoter. In some aspects, the Phas promoter comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 14.

In some aspects, the Phas promoter comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 14.

In some aspects, the recombinant sequences comprise a sequence named terminator that refers to nucleic acid sequences that define the end of a gene. Useful terminators include the following, but not limited to. Extensin terminator from tobacco (Rosenthal et al. (2018), Plant Mol. Biol. 96:429), Ub10 terminator from Arabidopsis thaliana (Tian et al. 2002, BIO-DES MANUF. 2022:1), Hsp70 terminator from Arabidopsis thaliana, (Tian et al. 2002, BIO-DES MANUF. 2022:1), Hsp18.2 terminator from Arabidopsis thaliana (Tian et al. 2002, BIO-DES MANUF. 2022:1), Act2 terminator from Arabidopsis thaliana (Tian et al. 2002, BIO-DES MANUF. 2022:1), G7 terminator from Arabidopsis thaliana (Tian et al. 2002, BIO-DES MANUF. 2022:1), 3g24240 terminator from Arabidopsis thaliana (Tian et al. 2002. BIO-DES MANUF. 2022:1), NOS terminator from Agrobacterium tumefaciens (Tian et al. 2002. BIO-DES MANUF. 2022:1) (SEQ ID NO: 10), Ocs terminator from Agrobacterium tumefaciens (Tian et al. 2002, BIO-DES MANUF. 2022:1), Mas terminator from Agrobacterium tumefaciens (Tian et al. 2002, BIO-DES MANUF. 2022:1), 35s terminator from Cauliflower Mosaic Virus (Tian et al. 2002, BIO-DES MANUF. 2022:1), Rbc terminator from Chrysanthemum (Tian et al. 2002, BIO-DES MANUF. 2022:1), Ags terminator from Agrobacterium tumefaciens (Tian et al. 2002, BIO-DES MANUF. 2022:1), 3′ utr-nos terminator from Agrobacterium tumefaciens (Tian et al. 2002, BIO-DES MANUF. 2022:1), 7s terminator from soybean (Tsubokura et al. (2012), Plant Mol. Biol. 78:301), E9 terminator from Pisum sativum (Coruzzi et al. (1984), EMBO Rep. 3:1671), ORF25 terminator from Agrobacterium tumefaciens (Barker et al. (1983), Plant Mol. Biol. 2:335). pinII terminator from Solanum tuberosum (Keil et al. (1986), Nucleic Acids Res. 14:5641). tml terminator from Agrobacterium tumefaciens (Barker et al. (1983), Plant Mol Biol. 2:335), Tr7 terminator from Agrobacterium tumefaciens (Dhaese et al. (1983). EMBO Rep. 2:419). In some aspects, the terminators are NOS (FIGS. 1, 3, 6, 8) (SEQ ID NO: 10) or arc5 (FIGS. 2, 4, 5, 7, 9, 10) (SEQ ID NO: 12). Arc5 terminator, from Phaseolus vulgaris, provides sequences to terminate transcription and to direct poly-adenylation of the mRNA (Goossens et al. (1999), Plant Physiol. 120:1095) but is also reported to enhance gene expression and contribute to seed specific expression.

In some aspects, the expression cassette comprises an NOS terminator sequence. In some aspects, the NOS terminator comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 10.

In some aspects, the NOS terminator comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 10.

In some aspects, the expression cassette comprises an arc5 terminator sequence. In some aspects, the arc5 terminator comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 12.

In some aspects, the arc5 terminator comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 12.

In some aspects, the recombinant sequence comprises a translational or transcriptional enhancer sequence. An example of a translation enhancer is the S′ UTR TEV (Tobacco Etch Virus Translational Enhancer) (SEQ ID NO: 9). The 5′ leader of the tobacco etch virus (TEV) is one of the better-studied potyvirus translational enhancers, it contains two cap-independent regulatory elements (CIREs) that fold into pseudoknots, which can independently enhance translation of the downstream transgene (Carrington & Freed. (1993) J. Virol., 64:1590). In some aspects, the recombinant sequences include a matrix attached region (MAR) as enhancers. The Rb7 MAR (SEQ ID NO: 13) is a DNA element shown to increase transgene expression in plants. The addition of the Rb7 MAR has been shown to strongly enhance protein production when added to most transcriptional terminators (Diamos & Mason, (2018), Plant Biotechnol. J. 16:1971). Furthermore, MARs can further improve the stability of transgene expression levels and may confer protection against transgene silencing (Vain et al. (1999), Plant J. 18:233). In some aspects, the arc5 terminator is fused to the Rb7 Matrix Attachment Region (MAR) that increases the likelihood and magnitude of transgene expression.

In some aspects, the expression cassette comprises a Rb7MAR enhancer. In some aspects, the Rb7MAR enhancer comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 13.

In some aspects, the Rb7MAR enhancer comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 13.

In some aspects, the expression cassette comprises a TEV enhancer. In some aspects, the TEV enhancer comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 9.

In some aspects, the TEV enhancer comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 9.

In some aspects, specific combinations of regulatory elements (i.e. promoters, terminators, and enhancers), lead to an enhanced expression of the heme protein in seeds above, e.g., 5%, 8%, and 10% tsp. In some aspects, the expression cassette comprises a seed-specific promoter such the 7S or phas, and a terminator such arc5 fused to the Rb7MAR enhancer. In some aspects, the combinations identified herein are:
i. p7S+cDNAHP+arc5+Rb7MAR
ii. p7S+TEV+cDNAHP+arc5+Rb7MAR
iii. PPhas+TEV+cDNAHP+arc5+Rb7MAR
iv. PPhas+cDNAHP+arc5+Rb7MAR

The cDNAHP identifies the cDNA for the heme protein. In some aspects, the heme proteins are animal derived heme proteins. In some aspects, the heme proteins are derived from metazoan. In some aspects, the heme proteins are derived from red meat (e.g., beef, pork, goat, and lamb), poultry (e.g., chicken and turkey), and seafood (e.g., fish, crustaceans, and mollusks). In some aspects, the animal derived heme protein is a myoglobin. In some aspects, the animal derived heme protein is a hemoglobin. It is routine for a person skilled in the art to replace orthologous sequences from other organisms, so the mere replacement of the recombinant protein is also in the scope of this disclosure.

In some aspects, the present disclosure also provides a polynucleotide comprising a nucleic acid encoding for a heme protein, wherein said nucleic acid is operatively linked to a seed-specific promoter selected from the group consisting of beta-conglycinin alpha subunit of the 7S storage (7s) promoter from soybean, the beta-phaseolin (Phas) promoter from common bean, USP promoter from Vicia faba, SBP promoter from Vicia faba. Legumin B4 promoter from Vicia faba, Napin promoter from Brassica napus, Vicilin promoter from Pisum sativum, α-globulin promoter from cotton, γ-zein promoter from maize, glutenin promoter from wheat, VvβVPE promoter from Vitis spp. Groundnut seed promoter (GSP) from peanut, 7αP promoter from soybean, AtLAC15 promoter from Arabidopsis thaliana. SSPs promoter from chickpea. Lectin promoter from soybean. Oleosin promoter from Brassica napus. AhLEC1A promoter from peanut, Glu-ID-1 promoter from wheat, Sesame 2S albumin (2Salb) promoter from sesame, and 8SGα promoter from mung bean.

In some aspects, said heme protein comprises a plant derived heme protein, a microorganism derived heme protein, or an animal derived heme protein.

In some aspects, said nucleic acid encoding for a heme protein comprises a nucleic acid sequence having at least 80% sequence identity to SEQ ID NO: 1 or SEQ NO: 2.

In some aspects, the polynucleotide comprises a nucleic acid sequence having at least 80% sequence identity to any one of SEQ ID NOs: 3 to 7.

In some aspects, said nucleic acid further comprises a transcription terminator selected from the group consisting of: Extensin terminator from tobacco, Ub10 terminator from Arabidopsis thaliana, Hsp70 terminator from Arabidopsis thaliana, Hsp18.2 terminator from Arabidopsis thaliana, Act2 terminator from Arabidopsis thaliana, G7 terminator from Arabidopsis thaliana, 3g24240 terminator from Arabidopsis thaliana. NOS terminator from Agrobacterium tumefaciens, Ocs terminator from Agrobacterium tumefaciens, Mas terminator from Agrobacterium tumefaciens, 35s terminator from Cauliflower Mosaic Virus, Rbc terminator from Chrysanthemum, Ags terminator from Agrobacterium tumefaciens, 3′ utr-nos terminator from Agrobacterium tumefaciens, 7s terminator from soybean, E9 terminator from Pisum sativum, ORF25 terminator from Agrobacterium tumefaciens, pinII terminator from Solanum tuberosum, tml terminator from Agrobacterium tumefaciens, Tr7 terminator from Agrobacterium tumefaciens, and the Arc5 terminator from Phaseolus vulgaris.

In some aspects, said nucleic acid further comprises a transcriptional or translational enhancer selected from the group consisting of 5′ UTR TEV and Rb7Mar 3′ Matrix Attachment Region.

In some aspects, the polynucleotide comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 1.

In some aspects, the polynucleotide comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 1.

In some aspects, the polynucleotide comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 2.

In some aspects, the polynucleotide comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 2.

In some aspects, the polynucleotide comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 3.

In some aspects, the polynucleotide comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 3.

In some aspects, the polynucleotide comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 4.

In some aspects, the polynucleotide comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 4.

In some aspects, the polynucleotide comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 5.

In some aspects, the polynucleotide comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 5.

In some aspects, the polynucleotide comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 6.

In some aspects, the polynucleotide comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 6.

In some aspects, the polynucleotide comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 7.

In some aspects, the polynucleotide comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 7.

In some aspects, the polynucleotide comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 8.

In some aspects, the polynucleotide comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 8.

In some aspects, the polynucleotide comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 9.

In some aspects, the polynucleotide comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 9.

In some aspects, the polynucleotide comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 10.

In some aspects, the polynucleotide comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 10.

In some aspects, the polynucleotide comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 11.

In some aspects, the polynucleotide comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 11.

In some aspects, the polynucleotide comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 12.

In some aspects, the polynucleotide comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 12.

In some aspects, the polynucleotide comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 13.

In some aspects, the polynucleotide comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 13.

In some aspects, the polynucleotide comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 14.

In some aspects, the polynucleotide comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 14.

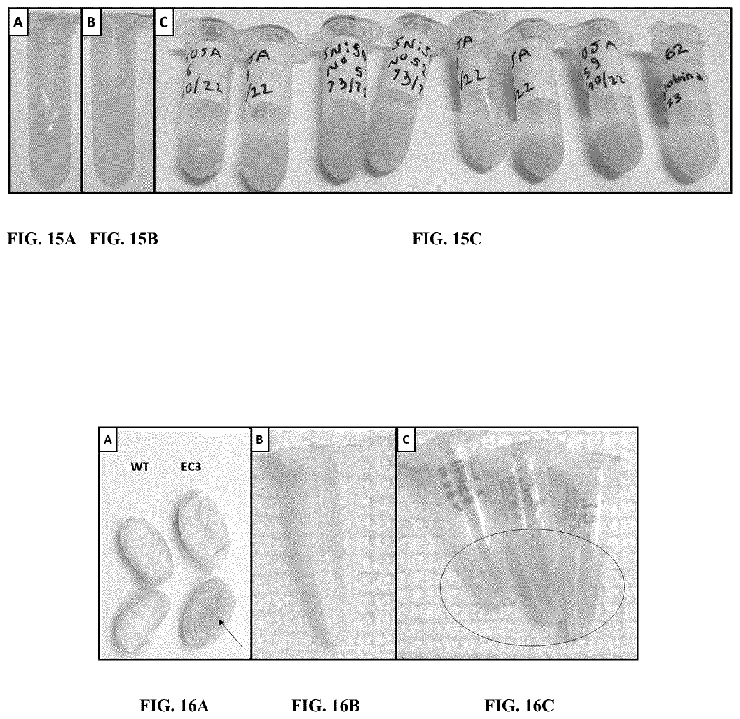

In some aspects, a plant is transformed with each of the expression cassettes (FIGS. 1-10). In some aspects, a stably transformed plant comprises in its genome: a recombinant DNA construct, wherein the heme protein is stably expressed and produces a pink color in the seed cotyledons and seed protein extracts (FIGS. 15A-15C and 16A-16C). The presence of heme proteins in transgenic organisms has resulted in visual color changes in protein extracts (pink color) when compared to WT (Carlsson et al. (2020). Sci. Rep. 10:1).

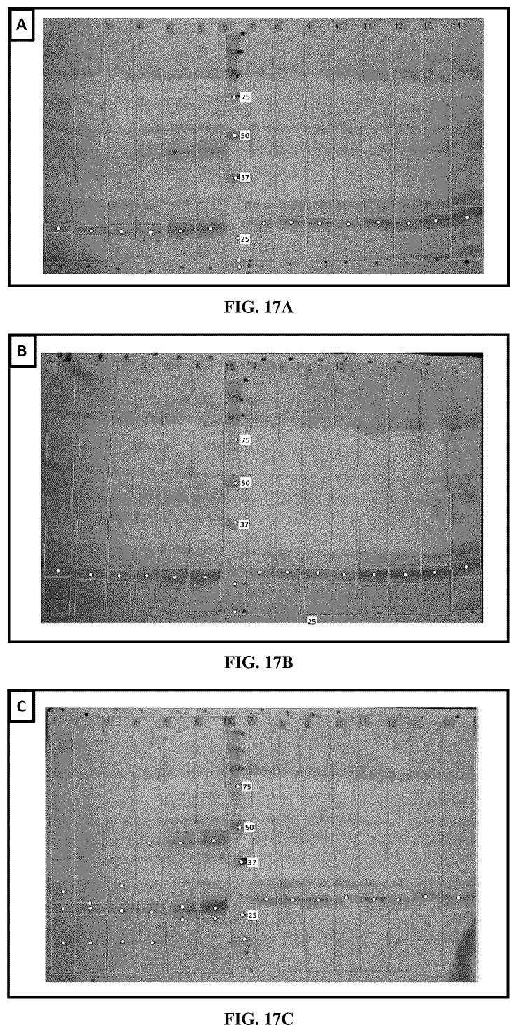

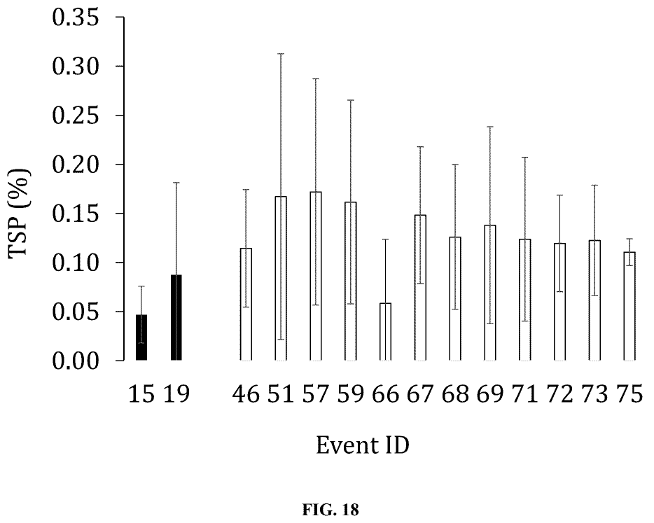

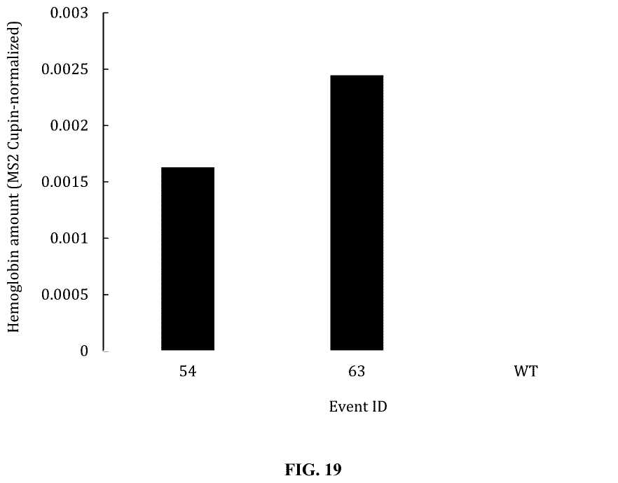

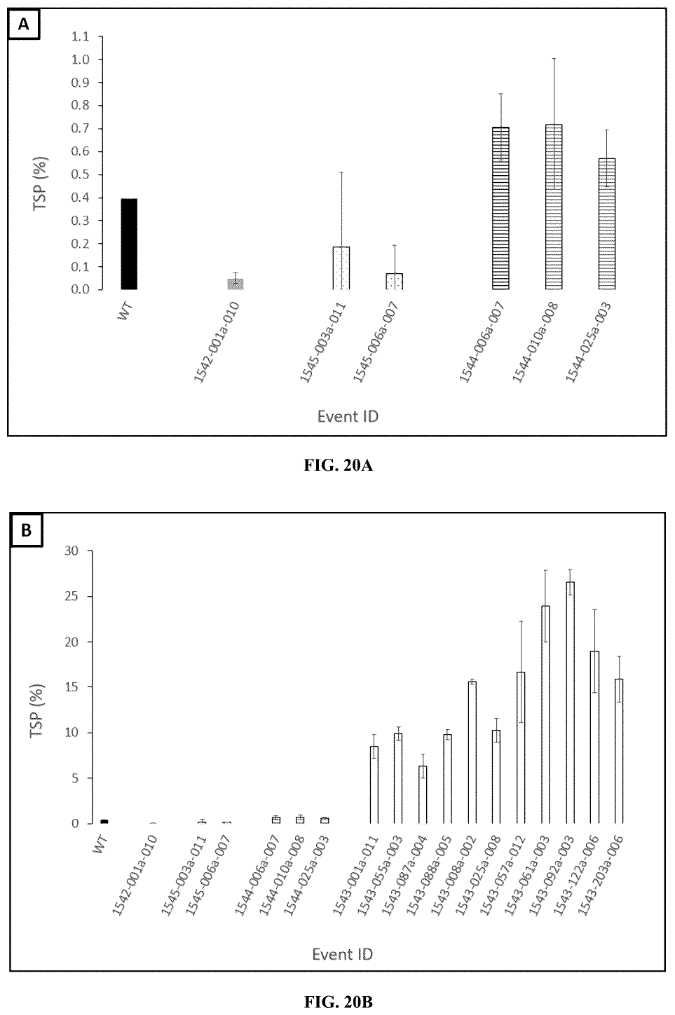

In some aspects, a stably transformed plant comprises in its genome: a recombinant DNA construct, wherein the heme protein is stably expressed, extracted via standard protein extractions protocols, and detected via Western Blot (FIGS. 17A-17C, 18). Liquid chromatography-mass spectrometry (LC-MS) (FIG. 19), and/or ELISA assays (FIGS. 20A-20B).

In some aspects, a stably transformed plant comprises in its genome: a recombinant DNA construct, wherein the heme protein is stably expressed in an amount of about 5% tsp or higher (FIG. 20B). In some aspects, the heme protein is stably expressed in an amount of about 8% tsp or higher (FIG. 20B). In some aspects, the heme protein is stably expressed in an amount of about 10% tsp or higher (FIG. 20B). In some aspects, the heme protein is stably expressed in an amount of about 25% or higher (FIG. 20B).

In some aspects, the recombinant heme proteins used for transformation are hemoglobin and myoglobin. In some aspects, the hemoglobin described herein is isolated from pig (Sus scrofa). In some aspects, the hemoglobin is a recombinant HbA-LL-HbB and it comprises the hemoglobin A subunit, a long linker, and the hemoglobin B subunit. In some aspects, the myoglobin described herein is isolated from pig (Sus scrofa domesticus). In some aspects, the expression cassette comprises any of the sequences disclosed in Table 1.

In some aspects, the expression cassette comprises SEQ ID NO: 11, SEQ ID NO: 12, and/or SEQ ID NO: 13. In some aspects, the expression cassette comprises SEQ ID NO: 11, SEQ ID NO: 9, SEQ ID NO: 12, and/or SEQ ID NO: 13. In some aspects, the expression cassette comprises SEQ ID NO: 14, SEQ ID NO: 9, SEQ ID NO: 12, and SEQ ID NO: 13. In some aspects, the expression cassette comprises SEQ ID NO: 14, SEQ ID NO: 12, and SEQ ID NO: 13.

In some aspects, the expression cassette comprises SEQ ID NO: 11, SEQ ID NO: 1, SEQ ID NO: 12, and/or SEQ ID NO: 13. In some aspects, the expression cassette comprises SEQ ID NO: 11, SEQ ID NO: 9, SEQ ID NO: 1, SEQ ID NO: 12, and/or SEQ ID NO. 13. In some aspects, the expression cassette comprises SEQ ID NO: 14, SEQ ID NO: 9. SEQ ID NO: 1, SEQ ID NO: 12, and SEQ ID NO: 13. In some aspects, the expression cassette comprises SEQ ID NO: 14, SEQ ID NO: 1, SEQ ID NO: 12, and SEQ ID NO: 13.

In some aspects, the expression cassette comprises SEQ ID NO: 11, SEQ ID NO: 2. SEQ ID NO: 12, and/or SEQ ID NO: 13. In some aspects, the expression cassette comprises SEQ ID NO: 11, SEQ ID NO: 9, SEQ ID NO: 2, SEQ ID NO: 12, and/or SEQ ID NO: 13. In some aspects, the expression cassette comprises SEQ ID NO: 14, SEQ ID NO: 9. SEQ ID NO: 2, SEQ ID NO: 12, and SEQ ID NO: 13. In some aspects, the expression cassette comprises SEQ ID NO: 14, SEQ ID NO: 2, SEQ ID NO: 12, and SEQ ID NO: 13.

In some aspects, provided herein is a transgenic plant, plant tissue, or plant cell comprising an expression cassette comprising an exogenous nucleic acid encoding for a heme protein. In some aspects, said nucleic acid is operatively linked to a seed-specific promoter and a transcription terminator. In some aspects, said heme protein is expressed in a seed in an amount of about 5% TSP. In some aspects, said heme protein is expressed in a seed in an amount of about 6% TSP. In some aspects, said heme protein is expressed in a seed in an amount of about 7% TSP. In some aspects, said heme protein is expressed in a seed in an amount of about 8% TSP. In some aspects, said heme protein is expressed in a seed in an amount of about 9% TSP. In some aspects, said heme protein is expressed in a seed in an amount of about 10% TSP. In some aspects, said heme protein is expressed in a seed in an amount of about 11% TSP. In some aspects, said heme protein is expressed in a seed in an amount of about 12% TSP. In some aspects, said heme protein is expressed in a seed in an amount of about 13% TSP. In some aspects, said heme protein is expressed in a seed in an amount of about 14% TSP. In some aspects, said heme protein is expressed in a seed in an amount of about 15% TSP. In some aspects, said heme protein is expressed in a seed in an amount of about 18% TSP. In some aspects, said heme protein is expressed in a seed in an amount of about 20% TSP. In some aspects, said heme protein is expressed in a seed in an amount of about 25% TSP. In some aspects, said heme protein is expressed in a seed in an amount of about 30% TSP. In some aspects, said heme protein is expressed in a seed in an amount between about 5% TSP and about 35% TSP. In some aspects, said heme protein is expressed in a seed in an amount between about 8% TSP and about 35% TSP. In some aspects, said heme protein is expressed in a seed in an amount between about 10% TSP and about 35% TSP. In some aspects, said heme protein is expressed in a seed in an amount between about 12% TSP and about 35% TSP. In some aspects, said heme protein is expressed in a seed in an amount between about 5% TSP and about 30% TSP. In some aspects, said heme protein is expressed in a seed in an amount between about 5% TSP and about 29% TSP. In some aspects, said heme protein is expressed in a seed in an amount between about 5% TSP and about 28% TSP. In some aspects, said heme protein is expressed in a seed in an amount between about 10% TSP and about 30% TSP. In some aspects, said heme protein is expressed in a seed in an amount between about 8% TSP and about 30% TSP. In some aspects, said heme protein is expressed in a seed in an amount between about 6% TSP and about 28% TSP.

In some aspects, the expression cassette comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 3.

In some aspects, the expression cassette comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 4.

In some aspects, the expression cassette comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 5.

In some aspects, the expression cassette comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 6.

In some aspects, the expression cassette comprises a nucleic acid sequence having at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 7.

In some aspects, the expression cassette comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 3.

In some aspects, the expression cassette comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 4.

In some aspects, the expression cassette comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 5.

In some aspects, the expression cassette comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 6.

In some aspects, the expression cassette comprises a nucleic acid sequence having about 80%, about 85%, about 90%, about 91%, about 92%, about 93%, about 94%, about 95%, about 96%, about 97%, about 98%, about 99%, or 100% sequence identity to SEQ ID NO: 7.

In some aspects, a stably transformed plant is soybean. Codon optimization is a process used to improve gene expression and increase translational efficiency of a gene of interest by accommodating codon bias of the host organism. In some aspects, the hemoglobin gene has been codon optimized for expression in soybean (SEQ ID NO: 1). In some aspects, the myoglobin gene has been codon optimized for expression in soybean (SEQ ID NO: 2).

In some aspects, the hemoglobin cDNA comprises a nucleic acid sequence having at least 70%, at least 75%, at least 80%, at least 85%, at least 90%, at least 95%, or at least 98% sequence identity to SEQ ID NO: 1.

In some aspects, the myoglobin cDNA comprises a nucleic acid sequence having at least 70%, at least 75%, at least 80%, at least 85%, at least 90%, at least 95%, or at least 98% sequence identity to SEQ ID NO: 2.

In some aspects, the hemoglobin cDNA comprises a nucleic acid sequence having about 70%, about 75%, about 80%, about 85%, about 90%, about 95%, or about 98% sequence identity to SEQ ID NO: 1.

In some aspects, the myoglobin cDNA comprises a nucleic acid sequence having about 70%, about 75%, about 80%, about 85%, about 90%, about 95%, about 98% sequence identity to SEQ ID NO: 2.

In some aspects, the recombinant sequences comprise a gene encoding for a selectable marker. In some aspects, the selectable marker is the BAR gene which produces the phosphinothricin N-acetyltransferase protein and provides resistance to gluphosinate. In some aspects, the BAR gene is located in the same plant transformation vector (circular plasmid) as the sequence of the heme protein (FIGS. 1-2, and 6-7). In some aspects, the selectable marker is the aadA gene which produces the aminoglycoside-3″-adenylyltransferase protein and provides resistance to aminoglycosides spectinomycin and streptomycin (FIGS. 3-5, and 8-10). In some aspects, the aadA gene is located in a separate linear construct and is co-bombarded with the linear construct holding the sequence of the heme proteins (FIGS. 3-5, and 8-10).

In some aspects, disclosed herein is a method to stably express a heme protein in plants, the method comprising a) transforming a plant with a plant transformation vector, b) regenerating the transgenic plants in vitro under selection pressure, and c) growing the transformed plants under the conditions wherein the recombinant heme proteins are expressed.

In some aspects, the levels of expression of a heme protein are referred to as “total soluble protein” (“TSP”). The expression level in TSP refers to an amount of a protein of interest relative to the total amount of protein that may reasonably be extracted from a plant using standard methods. Methods for extracting total protein from plant tissues such as seeds are known in the art (Cunha et al. (2011a), Transgenic Res. 20:811. Cunha et al. (2011b), Transgenic Res. 20:841, Ding et al. (2006), Biotechnol. Lett. 28:869). The amount of protein of interest may be measured using methods known in the art, such as an ELISA or a Western Blot.

The heme proteins and transgenic plants described herein may be used to prepare food compositions. In some aspects, the recombinant heme proteins produced by the transgenic plants may be used in its entirety, fractions and modifications thereof including solubilized, precipitated, partially or fully hydrolyzed, crosslinked, emulsified, texturized, cooked, extruded, reacted, structured versions to prepare meat-like (meat analogs) food stuffs including comminuted meats such as minced meat, meat strips, cubes and steaks; reconstituted and formed meat-like products including burgers, fillets, balls, sticks, slabs; reconstituted and stuffed/filled meat-like (meat analog) products including sausages, ham-like products, spreadables, reconstituted and coated meat-like products including nuggets, patties, strips, poppers, rings and more. The recombinant heme proteins may also be extracted from the transgenic plant using standard methods known in the field.

In some aspects, the food composition is prepared using the seed of the transgenic plant expressing the recombinant heme protein. In some aspects, the food composition is prepared using the recombinant heme protein extracted and purified from the seed.

The following experiments demonstrate different recombinant sequences that contain heme proteins and methods for producing recombinant proteins in plants. While the examples below describe expression in soybean, it will be understood by those skilled in the art that the expression sequences and methods disclosed herein may be tailored for expression in any monocot or dicot plants.

The practice of the present disclosure will employ, unless otherwise indicated, conventional techniques of cell biology, cell culture, molecular biology, transgenic biology, microbiology, recombinant DNA, biotechnology, plant genetic engineering and immunology, which are within the skill of the art. Such techniques are explained fully in the literature.

All of the references cited above, as well as all references cited herein, are incorporated herein by reference in their entireties.

The present disclosure is to be considered as an exemplification of the invention and is not intended to limit the invention to the specific aspects illustrated by the figures or description below. The present invention will now be described by referencing the appended figures representing specific aspects.

### EXAMPLES

### Example 1. Construction of Plant Transformation Vectors

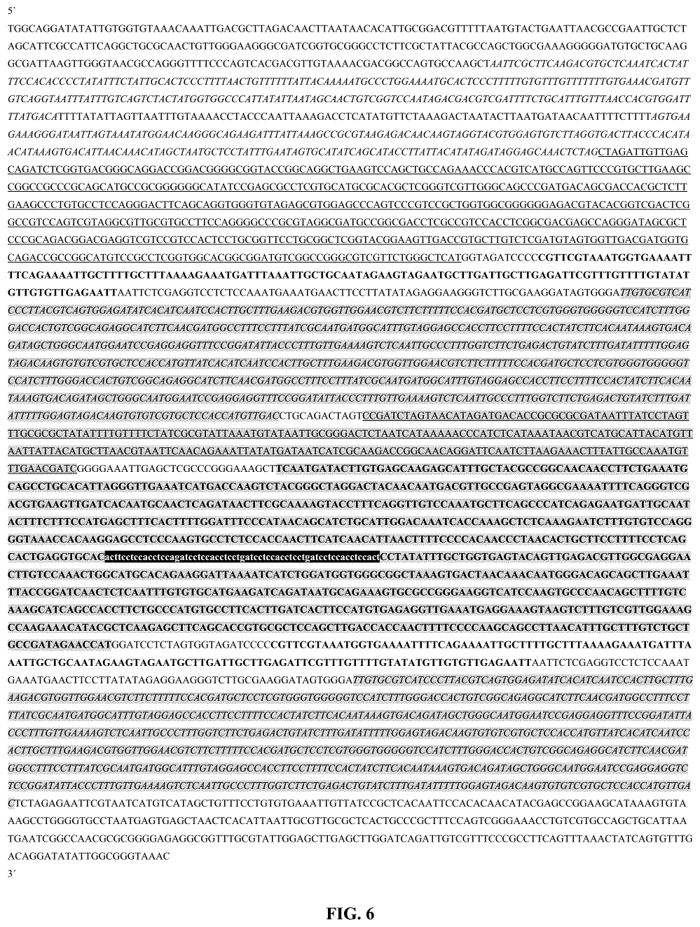

A codon-optimized gene comprising the alpha and beta subunits of porcine hemoglobin genes, referred to as HbA-LL-HbB, was synthesized by Genscript. The HbA-LL-HbB gene was cloned into the inhouse pIPTRA0-2×35S-MCS vector using the BamHI/HindIII restriction sites. The HbA-LL-HbB gene was cloned in between the 35S promoter and NOS terminator to create the pIPTRA0: p35S+HbA-LL-HbB vector (SEQ ID NO: 3). FIG. 1 shows a graphic representation of the pIPTRA0: p35S+HbA-LL-HbB vector, while FIG. 6 shows the nucleotide sequences for each of the regulatory elements involved.

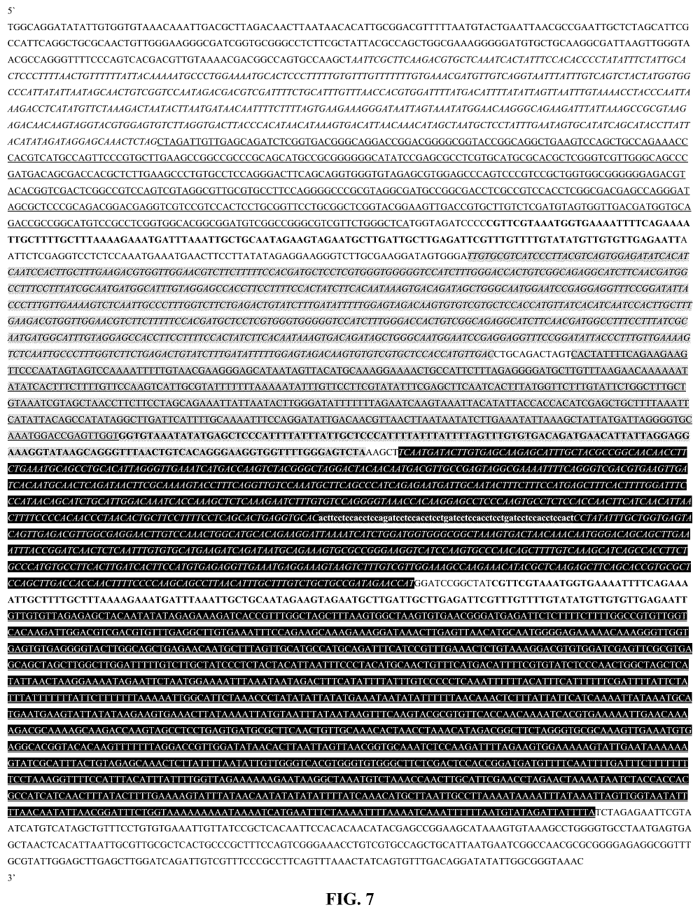

The 7S promoter fused to TEV Enhancer (p7S-TEV), and the arc5 Terminator fused to Rb7 Matrix Attachment Region (arc5T-Rb7MAR) were synthesized by Genscript. The pIPTRA0: p35S+HbA-LL-HbB vector was modified to create the pIPTRA0: p7S+HbA-LL-HbB-Arc5T vector (SEQ ID NO:4). The 35S promoter was replaced with the p7S-TEV using the XbaI/BamHI restriction sites. The NOS terminator was replaced by the arc5T-Rb7MAR fusion using the HindIII/SpeI restriction sites. The graphic representation and nucleotide sequences of pIPTRA0: p7S+HbA-LL-HbB-Arc5T vector are shown in FIGS. 2 and 7, respectively.

The 35S+TEV+myoglobincDNA+NOS (SEQ ID NO:5), p7S+TEV+myoglobincDNA+arc5+Rb7MAR (SEQ ID NO:6), and PPhas+myoglobincDNA+arc5+Rb7MAR (SEQ ID NO:7) expression vectors are referred to as EC1, EC2, and EC3, respectively. EC1, EC2, and EC3 were assembled via Golden Gate cloning in the in-house pEXPLODER plasmid. Promoters, myoglobin, and terminators were incorporated into the pEXPLODER plasmid. Following successful assembly of EC1, EC2, and EC3 linear fragments were released from the circular plasmid via BsaI digestion, followed by size separation in a 0.5% (w/v) agarose gel. After gel purification, EC1, EC2, and EC3 were separated from the section carrying the selectable marker via Asc1 digestion. The two resulting linear constructs (selectable marker+EC1, EC2, or EC3) were co-bombarded into soybean explants. Graphic representation of the three linearized fragments are presented in FIGS. 3, 4, and 5. Nucleotide sequences for EC1, EC2, and EC3 are presented in FIGS. 8, 9, and 10, respectively.

### Example 2. Confirmation of Transgenic Events

In vitro regeneration of putative transgenic lines was obtained for all the constructions used in this aspect (FIGS. 11A-11B). DNA was extracted from leaf tissue of regenerated explants for further genetic screening.

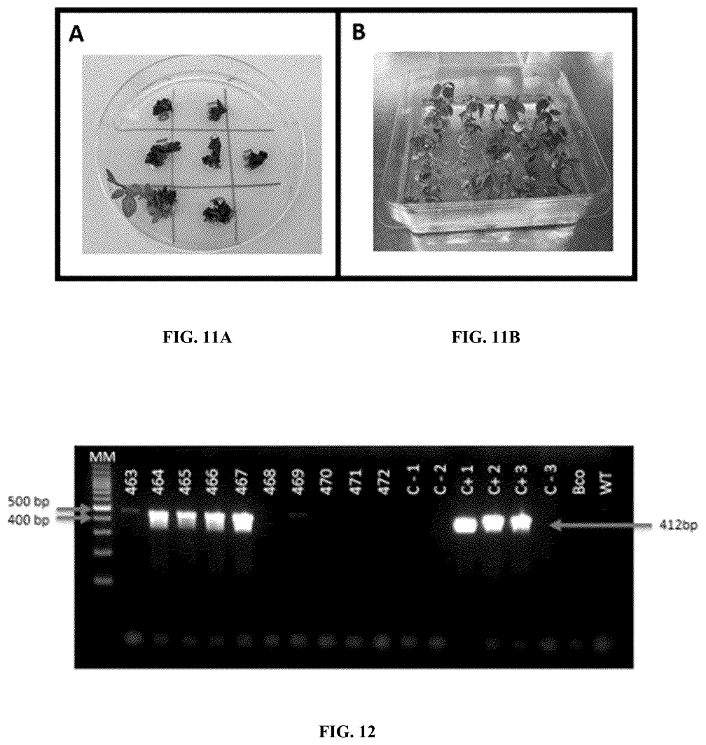

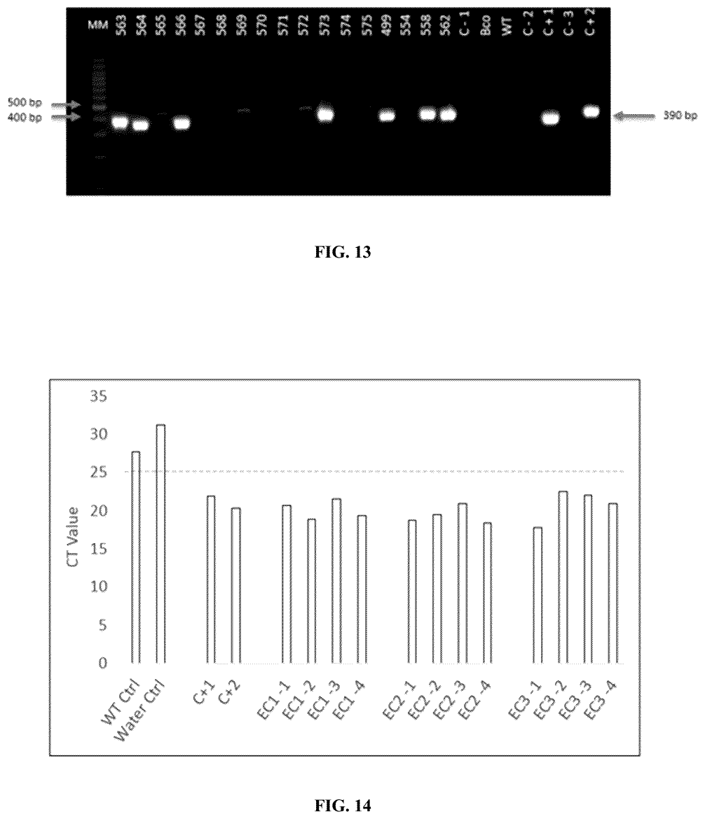

DNA from putative transformed lines with pIPTRA0: p35S+HbA-LL-HbB and pIPTRA0: p7S+HbA-LL-HbB-Arc5T was PCR-screened for the presence of the transgenic insert in the host genome. Agarose gel pictures show PCR amplification results for putative transgenic lines for pIPTRA0: p35S+HbA-LL-HbB (FIG. 12) and IPTRA0: p7S+HbA-LL-HbB-Arc5T (FIG. 13). The presence of a 412 and 390 bp band confirms the presence of the transgenic insert for lines transformed with pIPTRA0: p35S+HbA-LL-HbB and IPTRA0: p7S+HbA-LL-HbB-Arc5T, respectively.

qPCR of a section of the aadA1a CDS was performed in order to confirm the presence of the transgenic insert for lines putatively transformed with EC1, EC2, and EC3 (FIG. 14). The marker Ct cutoff for positive lines must be <25.

### Example 3. Total Soluble Protein Production in Soybean Transgenic Events Carrying the Porcine Hemoglobin Gene

The transgenic TO plants transformed with pIPTRA0: p35S+HbA-LL-HbB and pIPTRA0: p7S+HbA-LL-HbB-Arc5T were cultivated and propagated to T1 seeds. T1 seeds were screened for the presence of the porcine hemoglobin gene via PCR, a small section of the seed was excised for PCR purposes.

A total of 3 PCR positive seeds per transgenic event were pooled and protein extraction was performed. Pooled seeds were crushed in tissue-lyser, treated with extraction buffer (50 mM Tris-Cl pH 6.8, NaCl 50 mM, Na2SO3 36 mM, PHIC 1:200), and centrifuged at 13000 rpm at 4° C. for 10 minutes (FIGS. 15A-15C). After protein extraction, the extracts were then run on 10-well and 12% SDS PAGE gels, loading 100 μg proteins of each transgenic extract, a molecular weight standard, 100 μg protein extract of a WT used as a negative control, and WT extract+10, 25, 50, 150, and 250 ng of Hb standard (Sigma-Aldrich Hemoglobin porcine-lyophilized powder cat #H4131). Protein bands were then transferred to a nitrocellulose membrane and western blots were developed with the anti-porcine antibody (UsBiological Life sciences cat #140639) diluted 1/250 (FIGS. 17A-17C). Using the western blots, detected hemoglobin was quantified by comparing the intensity of the hemoglobin bands from the seed extracts with that from the hemoglobin standards, allowing calculation of the percentage hemoglobin of TSP (FIG. 18; Table 1).

TABLE 1
Accumulation of hemoglobin as a percentage of TSP
content in independent transgenic soybean seed stocks.
Table includes the coefficient of variation (%).

| | Event | Average | Coefficient of |
|---|---|---|---|
| Construct | ID | TSP (%) | Variation (%) |
| Empty Vector | 93 | <LOQ | n.d. |
| | 94 | <LOD | n.d. |
| pIPTRA0: | 14 | 0.036 | n.d. |
| p35S + HbA- | 15 | 0.047 | 0.238 |
| LL-HbB | 17 | <LOD | n.d. |
| | 18 | <LOD | n.d. |
| | 19 | 0.087 | 0.776 |
| pIPTRA0: | 46 | 0.114 | 0.495 |
| p7S + HbA-LL- | 47 | <LOD | n.d |
| HbB-Arc5T | 49 | 0.075 | n.d. |
| | 51 | 0.167 | 1.203 |
| | 52 | <LOD | n.d. |
| | 54 | 0.066 | 0.683 |
| | 57 | 0.172 | 0.952 |
| | 59 | 0.162 | 0.858 |
| | 61 | 0.039 | 0.930 |
| | 62 | 0.079 | 1.145 |
| | 63 | 0.078 | 0.406 |
| | 64 | 0.084 | 0.457 |
| | 65 | 0.110 | 0.320 |
| | 66 | 0.058 | 0.539 |
| | 67 | 0.148 | 0.576 |
| | 68 | 0.126 | 0.610 |
| | 69 | 0.138 | 0.829 |
| | 70 | 0.085 | 0.283 |
| | 71 | 0.124 | 0.690 |
| | 72 | 0.119 | 0.407 |
| | 73 | 0.122 | 0.466 |
| | 75 | 0.110 | 0.113 |
| | 76 | 0.059 | 0.036 |
| | 77 | 0.077 | 0.058 |
| | 81 | <LOQ | n.d. |
| | 83 | 0.038 | n.d. |
| | 84 | 0.074 | 0.654 |
| | 85 | 0.030 | n.d. |
| | 86 | <LOQ | n.d. |
| | 87 | 0.060 | n.d. |
| | 88 | 0.069 | n.d. |
| | 89 | 0.189 | n.d. |
| | 90 | <LOQ | n.d. |

“n.d”: Not determined, events with no coefficient of variation because two of three replicates were discarded.

Porcine hemoglobin identification and quantification from whole soybean seed protein extracts was also performed via Liquid chromatography-Mass spectrometry (LC-MS) (FIG. 18). An equal amount of soybean seed protein per transgenic event was digested with LysC/trypsin and then peptides desalted on C18 tips. LC-MS data were acquired on a Bruker timsTOF-Pro2 and searched against a soybean database supplemented with the pig hemoglobin sequence provided. The amounts of hemoglobin were determined by normalizing its MS2 intensity to soybean cupin. No hemoglobin was detected in the WT sample.

Transgenic events carrying the porcine myoglobin gene: The transgenic TO plants transformed with EC1, EC2, and EC3 linear constructs were cultivated and propagated up to T2 seeds. T2 seeds were screened for the presence of the porcine myoglobin gene via ddPCR; a small section of the seed was excised for PCR purposes.

A total of 3 PCR positive seeds per transgenic event were pooled and protein extraction was performed. Pooled seeds were ground in extraction buffer (5% w/v SDS, 175 mM Tris-HCl, pH 8.0, 0.4% v/v beta-mercaptoethanol) with Omni ceramic beads (1.4 mm); the extracts were heated to 65° C. for 25 min, centrifuged and the supernatants were transferred to fresh tubes. Myoglobin quantitation from seed extracts was done using the Alpha Diagnostics ELISA kit (cat #600-640-PMY). All samples were normalized to 50 μg/mL total soluble protein (TSP) and tested for myoglobin content according to the manufacturer's protocols. The concentration of myoglobin was determined by reference to the standard curve. Each event consisted of one biological replicate (a pool of 3 seeds) with 3 technical replicates. Twenty μL (1 μg) was tested for each sample. The percentage myoglobin of TSP is presented in FIGS. 20A-20B and Table 2.

TABLE 2
Accumulation of myoglobin as a percentage of TSP content
(±s.d.) in independent transgenic soybean seed stocks.
TSP content above 5% are shaded in gray.

| Linear | | Average | |
|---|---|---|---|
| Construct | Event ID | TSP (%) | SD |
| WT | WT | 0.40 | 0.00 |
| Empty vector | 1542-001a-010 | 0.05 | 0.02 |
| EC1 | 1545-003a-011 | 0.19 | 0.32 |
| | 1545-004a-006 | 0.00 | 0.00 |
| | 1545-006a-007 | 0.07 | 0.12 |
| | 1545-013a-004 | 0.42 | 0.06 |
| | 1545-016a-008 | 0.00 | 0.01 |
| | 1545-024a-002 | 1.43 | 0.19 |
| | 1545-033a-009 | 0.00 | 0.01 |
| | 1545-034a-003 | 0.01 | 0.01 |
| | 1545-036a-008 | 0.42 | 0.10 |
| | 1545-037a-011 | 0.01 | 0.04 |
| EC2 | 1544-002a-006 | 0.09 | 0.13 |
| | 1544-006a-007 | 0.71 | 0.15 |
| | 1544-010a-008 | 0.72 | 0.29 |
| | 1544-011a-010 | 0.04 | 0.01 |
| | 1544-013a-008 | 0.01 | 0.01 |
| | 1544-014a-002 | 0.00 | 0.01 |
| | 1544-015a-010 | 0.04 | 0.03 |
| | 1544-016a-008 | 0.01 | 0.00 |
| | 1544-017a-010 | 4.19 | 2.32 |
| | 1544-005a-002 | 0.00 | 0.00 |
| | 1544-018a-003 | 0.19 | 0.18 |
| | 1544-022a-007 | 0.18 | 0.34 |
| | 1544-023a-007 | 0.00 | 0.01 |
| | 1544-025a-003 | 0.57 | 0.13 |
| | 1544-027a-001 | 0.43 | 0.39 |
| | 1544-030a-001 | 0.21 | 0.12 |
| EC3 | 1543-001a-011 | 8.47 | 1.29 |
| | 1543-033a-010 | 3.01 | 1.58 |
| | 1543-043a-006 | 0.79 | 0.10 |
| | 1543-051a-003 | 0.46 | 0.02 |
| | 1543-055a-003 | 9.93 | 0.76 |
| | 1543-059a-009 | 8.01 | 1.59 |
| | 1543-064a-007 | 1.31 | 0.13 |
| | 1543-068a-001 | 0.43 | 0.01 |
| | 1543-087a-004 | 6.34 | 1.29 |
| | 1543-088a-005 | 9.81 | 0.53 |
| | 1543-095a-009 | 1.54 | 0.22 |
| | 1543-004a-008 | 1.72 | 0.36 |
| | 1543-005a-010 | 0.03 | 0.01 |
| | 1543-006a-002 | 0.19 | 0.07 |
| | 1543-007a-004 | 0.13 | 0.16 |
| | 1543-008a-002 | 15.59 | 0.26 |
| | 1543-009a-001 | 0.03 | 0.02 |
| | 1543-011a-002 | 10.59 | 3.28 |
| | 1543-012a-008 | 0.11 | 0.08 |
| | 1543-016a-001 | 0.17 | 0.11 |
| | 1543-017a-005 | 0.04 | 0.00 |
| | 1543-023a-005 | 0.02 | 0.01 |
| | 1543-025a-008 | 10.24 | 1.33 |
| | 1543-046a-001 | 0.11 | 0.02 |
| | 1543-053a-004 | 0.07 | 0.02 |
| | 1543-054a-007 | 0.24 | 0.02 |
| | 1543-057a-012 | 16.67 | 5.57 |
| | 1543-058a-012 | 0.30 | 0.36 |
| | 1543-061a-003 | 23.94 | 3.95 |
| | 1543-071a-005 | 1.01 | 0.42 |
| | 1543-073a-004 | 0.43 | 0.65 |
| | 1543-078a-001 | 0.06 | 0.01 |
| | 1543-085a-007 | 4.29 | 1.32 |
| | 1543-092a-003 | 26.58 | 1.43 |
| | 1543-122a-006 | 19.02 | 4.59 |
| | 1543-131a-004 | 2.55 | 0.65 |
| | 1543-134a-002 | 0.29 | 0.18 |
| | 1543-153a-008 | 3.16 | 2.58 |
| | 1543-183a-001 | 7.35 | 2.09 |
| | 1543-191a-006 | 15.82 | 2.52 |
| | 1543-203a-006 | 15.88 | 2.56 |
| | 1543-116a-004 | 0.00 | 0.00 |
| | 1543-119a-007 | 0.46 | 0.58 |
| | 1543-126a-008 | 0.03 | 0.02 |
| | 1543-132a-001 | 0.00 | 0.00 |
| | 1543-133a-009 | 1.05 | 1.46 |
| | 1543-137a-008 | 0.07 | 0.07 |
| | 1543-143a-002 | 7.73 | 6.88 |
| | 1543-165a-001 | 2.11 | 0.77 |

### Sequences

| SEQ ID | Brief reference | nt |
|---|---|---|
| SEQ ID | porcine | TCAATGATACTTGTGAGCAAGAGCATTTGCTAC |
| NO: 1 | hemoglobin | GCCGGCAACAACCTTCTGAAATGCAGCCTGCAC |
| | (HbALLHbB) | ATTAGGGTTGAAATCATGACCAAGTCTACGGGC |
| | | TAGGACTACAACAATGACGTTGCCGAGTAGGCG |
| | | AAAATTTTCAGGGTCGACGTGAAGTTGATCACA |
| | | ATGCAACTCAGATAACTTCGCAAAAGTACCTTTC |
| | | AGGTTGTCCAAATGCTTCAGCCCATCAGAGAAT |
| | | GATTGCAATACTTTCTTTCCATGAGCTTTCACTT |
| | | TTGGATTTCCCATAACAGCATCTGCATTGGACAA |
| | | ATCACCAAAGCTCTCAAAGAATCTTTGTGTCCAG |
| | | GGGTAAACCACAAGGAGCCTCCCAAGTGCCTCT |
| | | CCACCAACTTCATCAACATTAACTTTTCCCCACA |
| | | ACCCTAACACTGCTTCCTTTTCCTCAGCACTGAG |
| | | GTGCACACTTCCTCCACCTCCAGATCCTCCACCT |
| | | CCTGATCCTCCACCTCCTGATCCTCCACCTCCAC |
| | | TCCTATATTTGCTGGTGAGTACAGTTGAGACGTT |
| | | GGCGAGGAACTTGTCCAAACTGGCATGCACAGA |
| | | AGGATTAAAATCATCTGGATGGTGGGCGGCTAA |
| | | AGTGACTAACAAACAATGGGACAGCAGCTTGAA |
| | | ATTTACCGGATCAACTCTCAATTTGTGTGCATGA |
| | | AGATCAGATAATGCAGAAAGTGCGCCGGGAAGG |
| | | TCATCCAAGTGCCCAACAGCTTTTGTCAAAGCAT |
| | | CAGCCACCTTCTGCCCATGTGCCTTCACTTGATC |
| | | ACTTCCATGTGAGAGGTTGAAATGAGGAAAGTA |
| | | AGTCTTTGTCGTTGGAAAGCCAAGAAACATACG |
| | | CTCAAGAGCTTCAGCACCGTGCGCTCCAGCTTG |
| | | ACCACCAACTTTTCCCCAAGCAGCCTTAACATTT |
| | | GCTTTGTCTGCTGCCGATAGAACCAT |
| SEQ ID | porcine | ATGGGGCTATCAGATGGTGAATGGCAACTTGTA |
| NO: 2 | Myoglobin | TTGAATGTTTGGGGAAAAGTTGAAGCTGATGTT |
| | | GCTGGACATGGTCAAGAAGTGTTAATAAGACTC |
| | | TTCAAAGGCCACCCTGAAACATTAGAGAAGTTT |
| | | GACAAATTCAAGCACCTAAAATCTGAAGATGAA |
| | | ATGAAGGCCTCCGAGGACTTGAAGAAGCATGGA |
| | | AACACTGTCCTGACTGCACTCGGCGGGATCCTC |
| | | AAAAAGAAAGGTCATCATGAAGCGGAGTTGACA |
| | | CCATTGGCTCAGTCTCATGCTACCAAACACAAG |
| | | ATTCCTGTGAAGTATCTTGAGTTTATTAGTGAGG |
| | | CCATAATTCAGGTTTTGCAATCAAAACATCCCGG |
| | | TGATTTTGGTGCAGATGCTCAAGGAGCAATGAG |
| | | CAAAGCACTGGAGCTTTTCAGGAATGATATGGC |
| | | AGCCAAGTACAAGGAACTTGGATTTCAGGGGTG |
| | | A |
| SEQ ID | pIPTRA0:p35S + | 5′ |
| NO: 3 | HbA-LL-HbB | TGGCAGGATATATTGTGGTGTAAACAAATTGAC |
| | | GCTTAGACAACTTAATAACACATTGCGGACGTTT |
| | | TTAATGTACTGAATTAACGCCGAATTGCTCTAGC |
| | | ATTCGCCATTCAGGCTGCGCAACTGTTGGGAAG |
| | | GGCGATCGGTGCGGGCCTCTTCGCTATTACGCC |
| | | AGCTGGCGAAAGGGGGATGTGCTGCAAGGCGA |
| | | TTAAGTTGGGTAACGCCAGGGTTTTCCCAGTCA |
| | | CGACGTTGTAAAACGACGGCCAGTGCCAAGCTA |
| | | ATTCGCTTCAAGACGTGCTCAAATCACTATTTCC |
| | | ACACCCCTATATTTCTATTGCACTCCCTTTTAAC |
| | | TGTTTTTTATTACAAAAATGCCCTGGAAAATGCA |
| | | CTCCCTTTTTGTGTTTGTTTTTTTGTGAAACGAT |
| | | GTTGTCAGGTAATTTATTTGTCAGTCTACTATGG |
| | | TGGCCCATTATATTAATAGCAACTGTCGGTCCAA |
| | | TAGACGACGTCGATTTTCTGCATTTGTTTAACCA |
| | | CGTGGATTTTATGACATTTTATATTAGTTAATTT |
| | | GTAAAACCTACCCAATTAAAGACCTCATATGTTC |
| | | TAAAGACTAATACTTAATGATAACAATTTTCTTT |
| | | TAGTGAAGAAAGGGATAATTAGTAAATATGGAA |
| | | CAAGGGCAGAAGATTTATTAAAGCCGCGTAAGA |
| | | GACAACAAGTAGGTACGTGGAGTGTCTTAGGTG |
| | | ACTTACCCACATAACATAAAGTGACATTAACAAA |
| | | CATAGCTAATGCTCCTATTTGAATAGTGCATATC |
| | | AGCATACCTTATTACATATAGATAGGAGCAAACT |
| | | CTAGCTAGATTGTTGAGCAGATCTCGGTGACGG |
| | | GCAGGACCGGACGGGGCGGTACCGGCAGGCTG |
| | | AAGTCCAGCTGCCAGAAACCCACGTCATGCCAG |
| | | TTCCCGTGCTTGAAGCCGGCCGCCCGCAGCATG |
| | | CCGCGGGGGGCATATCCGAGCGCCTCGTGCATG |
| | | CGCACGCTCGGGTCGTTGGGCAGCCCGATGACA |
| | | GCGACCACGCTCTTGAAGCCCTGTGCCTCCAGG |
| | | GACTTCAGCAGGTGGGTGTAGAGCGTGGAGCCC |
| | | AGTCCCGTCCGCTGGTGGCGGGGGGAGACGTA |
| | | CACGGTCGACTCGGCCGTCCAGTCGTAGGCGTT |
| | | GCGTGCCTTCCAGGGGCCCGCGTAGGCGATGCC |
| | | GGCGACCTCGCCGTCCACCTCGGCGACGAGCCA |
| | | GGGATAGCGCTCCCGCAGACGGACGAGGTCGTC |
| | | CGTCCACTCCTGCGGTTCCTGCGGCTCGGTACG |
| | | GAAGTTGACCGTGCTTGTCTCGATGTAGTGGTT |
| | | GACGATGGTGCAGACCGCCGGCATGTCCGCCTC |
| | | GGTGGCACGGCGGATGTCGGCCGGGCGTCGTT |
| | | CTGGGCTCATGGTAGATCCCCCGTTCGTAAATG |
| | | GTGAAAATTTTCAGAAAATTGCTTTTGCTTTAAA |
| | | AGAAATGATTTAAATTGCTGCAATAGAAGTAGA |
| | | ATGCTTGATTGCTTGAGATTCGTTTGTTTTGTAT |
| | | ATGTTGTGTTGAGAATTAATTCTCGAGGTCCTCT |
| | | CCAAATGAAATGAACTTCCTTATATAGAGGAAG |
| | | GGTCTTGCGAAGGATAGTGGGATTGTGCGTCAT |
| | | CCCTTACGTCAGTGGAGATATCACATCAATCCA |
| | | CTTGCTTTGAAGACGTGGTTGGAACGTCTTCTTT |
| | | TTCCACGATGCTCCTCGTGGGTGGGGGTCCATC |
| | | TTTGGGACCACTGTCGGCAGAGGCATCTTCAAC |
| | | GATGGCCTTTCCTTTATCGCAATGATGGCATTTG |
| | | TAGGAGCCACCTTCCTTTTCCACTATCTTCACAA |
| | | TAAAGTGACAGATAGCTGGGCAATGGAATCCGA |
| | | GGAGGTTTCCGGATATTACCCTTTGTTGAAAAG |
| | | TCTCAATTGCCCTTTGGTCTTCTGAGACTGTATC |
| | | TTTGATATTTTTGGAGTAGACAAGTGTGTCGTGC |
| | | TCCACCATGTTATCACATCAATCCACTTGCTTTG |
| | | AAGACGTGGTTGGAACGTCTTCTTTTTCCACGAT |
| | | GCTCCTCGTGGGTGGGGGTCCATCTTTGGGACC |
| | | ACTGTCGGCAGAGGCATCTTCAACGATGGCCTT |
| | | TCCTTTATCGCAATGATGGCATTTGTAGGAGCC |
| | | ACCTTCCTTTTCCACTATCTTCACAATAAAGTGA |
| | | CAGATAGCTGGGCAATGGAATCCGAGGAGGTTT |
| | | CCGGATATTACCCTTTGTTGAAAAGTCTCAATTG |
| | | CCCTTTGGTCTTCTGAGACTGTATCTTTGATATT |
| | | TTTGGAGTAGACAAGTGTGTCGTGCTCCACCAT |
| | | GTTGACCTGCAGACTAGTCCGATCTAGTAACAT |
| | | AGATGACACCGCGCGCGATAATTTATCCTAGTT |
| | | TGCGCGCTATATTTTGTTTTCTATCGCGTATTAA |
| | | ATGTATAATTGCGGGACTCTAATCATAAAAACCC |
| | | ATCTCATAAATAACGTCATGCATTACATGTTAAT |
| | | TATTACATGCTTAACGTAATTCAACAGAAATTAT |
| | | ATGATAATCATCGCAAGACCGGCAACAGGATTC |
| | | AATCTTAAGAAACTTTATTGCCAAATGTTTGAAC |
| | | GATCGGGGAAATTGAGCTCGCCCGGGAAAGCTT |
| | | CAATGATACTTGTGAGCAAGAGCATTTGCTACG |
| | | CCGGCAACAACCTTCTGAAATGCAGCCTGCACA |
| | | TTAGGGTTGAAATCATGACCAAGTCTACGGGCT |
| | | AGGACTACAACAATGACGTTGCCGAGTAGGCGA |
| | | AAATTTTCAGGGTCGACGTGAAGTTGATCACAA |
| | | TGCAACTCAGATAACTTCGCAAAAGTACCTTTCA |
| | | GGTTGTCCAAATGCTTCAGCCCATCAGAGAATG |
| | | ATTGCAATACTTTCTTTCCATGAGCTTTCACTTT |
| | | TGGATTTCCCATAACAGCATCTGCATTGGACAA |
| | | ATCACCAAAGCTCTCAAAGAATCTTTGTGTCCAG |
| | | GGGTAAACCACAAGGAGCCTCCCAAGTGCCTCT |
| | | CCACCAACTTCATCAACATTAACTTTTCCCCACA |
| | | ACCCTAACACTGCTTCCTTTTCCTCAGCACTGAG |
| | | GTGCACACTTCCTCCACCTCCAGATCCTCCACCT |
| | | CCTGATCCTCCACCTCCTGATCCTCCACCTCCAC |
| | | TCCTATATTTGCTGGTGAGTACAGTTGAGACGTT |
| | | GGCGAGGAACTTGTCCAAACTGGCATGCACAGA |
| | | AGGATTAAAATCATCTGGATGGTGGGCGGCTAA |
| | | AGTGACTAACAAACAATGGGACAGCAGCTTGAA |
| | | ATTTACCGGATCAACTCTCAATTTGTGTGCATGA |
| | | AGATCAGATAATGCAGAAAGTGCGCCGGGAAGG |
| | | TCATCCAAGTGCCCAACAGCTTTTGTCAAAGCAT |
| | | CAGCCACCTTCTGCCCATGTGCCTTCACTTGATC |
| | | ACTTCCATGTGAGAGGTTGAAATGAGGAAAGTA |
| | | AGTCTTTGTCGTTGGAAAGCCAAGAAACATACG |
| | | CTCAAGAGCTTCAGCACCGTGCGCTCCAGCTTG |
| | | ACCACCAACTTTTCCCCAAGCAGCCTTAACATTT |
| | | GCTTTGTCTGCTGCCGATAGAACCATGGATCCT |
| | | CTAGTGGTAGATCCCCCGTTCGTAAATGGTGAA |
| | | AATTTTCAGAAAATTGCTTTTGCTTTAAAAGAAA |
| | | TGATTTAAATTGCTGCAATAGAAGTAGAATGCTT |
| | | GATTGCTTGAGATTCGTTTGTTTTGTATATGTTG |
| | | TGTTGAGAATTAATTCTCGAGGTCCTCTCCAAAT |
| | | GAAATGAACTTCCTTATATAGAGGAAGGGTCTT |
| | | GCGAAGGATAGTGGGATTGTGCGTCATCCCTTA |
| | | CGTCAGTGGAGATATCACATCAATCCACTTGCTT |
| | | TGAAGACGTGGTTGGAACGTCTTCTTTTTCCAC |
| | | GATGCTCCTCGTGGGTGGGGGTCCATCTTTGGG |
| | | ACCACTGTCGGCAGAGGCATCTTCAACGATGGC |
| | | CTTTCCTTTATCGCAATGATGGCATTTGTAGGAG |
| | | CCACCTTCCTTTTCCACTATCTTCACAATAAAGT |
| | | GACAGATAGCTGGGCAATGGAATCCGAGGAGGT |
| | | TTCCGGATATTACCCTTTGTTGAAAAGTCTCAAT |
| | | TGCCCTTTGGTCTTCTGAGACTGTATCTTTGATA |
| | | TTTTTGGAGTAGACAAGTGTGTCGTGCTCCACC |
| | | ATGTTATCACATCAATCCACTTGCTTTGAAGACG |
| | | TGGTTGGAACGTCTTCTTTTTCCACGATGCTCCT |
| | | CGTGGGTGGGGGTCCATCTTTGGGACCACTGTC |
| | | GGCAGAGGCATCTTCAACGATGGCCTTTCCTTT |
| | | ATCGCAATGATGGCATTTGTAGGAGCCACCTTC |
| | | CTTTTCCACTATCTTCACAATAAAGTGACAGATA |
| | | GCTGGGCAATGGAATCCGAGGAGGTCTCCGGAT |
| | | ATTACCCTTTGTTGAAAAGTCTCAATTGCCCTTT |
| | | GGTCTTCTGAGACTGTATCTTTGATATTTTTGGA |
| | | GTAGACAAGTGTGTCGTGCTCCACCATGTTGAC |
| | | TCTAGAGAATTCGTAATCATGTCATAGCTGTTTC |
| | | CTGTGTGAAATTGTTATCCGCTCACAATTCCACA |
| | | CAACATACGAGCCGGAAGCATAAAGTGTAAAGC |
| | | CTGGGGTGCCTAATGAGTGAGCTAACTCACATT |
| | | AATTGCGTTGCGCTCACTGCCCGCTTTCCAGTC |
| | | GGGAAACCTGTCGTGCCAGCTGCATTAATGAAT |
| | | CGGCCAACGCGCGGGGAGAGGCGGTTTGCGTA |
| | | TTGGAGCTTGAGCTTGGATCAGATTGTCGTTTC |
| | | CCGCCTTCAGTTTAAACTATCAGTGTTTGACAGG |
| | | ATATATTGGCGGGTAAAC |
| | | 3′ |
| SEQ ID | pIPTRA0:p7S + | 5′ |
| NO: 4 | HbA-LL-HbB-Arc5T | TGGCAGGATATATTGTGGTGTAAACAAATTGAC |
| | | GCTTAGACAACTTAATAACACATTGCGGACGTTT |
| | | TTAATGTACTGAATTAACGCCGAATTGCTCTAGC |
| | | ATTCGCCATTCAGGCTGCGCAACTGTTGGGAAG |
| | | GGCGATCGGTGCGGGCCTCTTCGCTATTACGCC |
| | | AGCTGGCGAAAGGGGGATGTGCTGCAAGGCGA |
| | | TTAAGTTGGGTAACGCCAGGGTTTTCCCAGTCA |
| | | CGACGTTGTAAAACGACGGCCAGTGCCAAGCTA |
| | | ATTCGCTTCAAGACGTGCTCAAATCACTATTTCC |
| | | ACACCCCTATATTTCTATTGCACTCCCTTTTAAC |
| | | TGTTTTTTATTACAAAAATGCCCTGGAAAATGCA |
| | | CTCCCTTTTTGTGTTTGTTTTTTTGTGAAACGAT |
| | | GTTGTCAGGTAATTTATTTGTCAGTCTACTATGG |
| | | TGGCCCATTATATTAATAGCAACTGTCGGTCCAA |
| | | TAGACGACGTCGATTTTCTGCATTTGTTTAACCA |
| | | CGTGGATTTTATGACATTTTATATTAGTTAATTT |
| | | GTAAAACCTACCCAATTAAAGACCTCATATGTTC |
| | | TAAAGACTAATACTTAATGATAACAATTTTCTTT |
| | | TAGTGAAGAAAGGGATAATTAGTAAATATGGAA |
| | | CAAGGGCAGAAGATTTATTAAAGCCGCGTAAGA |
| | | GACAACAAGTAGGTACGTGGAGTGTCTTAGGTG |
| | | ACTTACCCACATAACATAAAGTGACATTAACAAA |
| | | CATAGCTAATGCTCCTATTTGAATAGTGCATATC |
| | | AGCATACCTTATTACATATAGATAGGAGCAAACT |
| | | CTAGCTAGATTGTTGAGCAGATCTCGGTGACGG |
| | | GCAGGACCGGACGGGGCGGTACCGGCAGGCTG |
| | | AAGTCCAGCTGCCAGAAACCCACGTCATGCCAG |
| | | TTCCCGTGCTTGAAGCCGGCCGCCCGCAGCATG |
| | | CCGCGGGGGGCATATCCGAGCGCCTCGTGCATG |
| | | CGCACGCTCGGGTCGTTGGGCAGCCCGATGACA |
| | | GCGACCACGCTCTTGAAGCCCTGTGCCTCCAGG |
| | | GACTTCAGCAGGTGGGTGTAGAGCGTGGAGCCC |
| | | AGTCCCGTCCGCTGGTGGCGGGGGGAGACGTA |
| | | CACGGTCGACTCGGCCGTCCAGTCGTAGGCGTT |
| | | GCGTGCCTTCCAGGGGCCCGCGTAGGCGATGCC |
| | | GGCGACCTCGCCGTCCACCTCGGCGACGAGCCA |
| | | GGGATAGCGCTCCCGCAGACGGACGAGGTCGTC |
| | | CGTCCACTCCTGCGGTTCCTGCGGCTCGGTACG |
| | | GAAGTTGACCGTGCTTGTCTCGATGTAGTGGTT |
| | | GACGATGGTGCAGACCGCCGGCATGTCCGCCTC |
| | | GGTGGCACGGCGGATGTCGGCCGGGCGTCGTT |
| | | CTGGGCTCATGGTAGATCCCCCGTTCGTAAATG |
| | | GTGAAAATTTTCAGAAAATTGCTTTTGCTTTAAA |
| | | AGAAATGATTTAAATTGCTGCAATAGAAGTAGA |
| | | ATGCTTGATTGCTTGAGATTCGTTTGTTTTGTAT |
| | | ATGTTGTGTTGAGAATTAATTCTCGAGGTCCTCT |
| | | CCAAATGAAATGAACTTCCTTATATAGAGGAAG |
| | | GGTCTTGCGAAGGATAGTGGGATTGTGCGTCAT |
| | | CCCTTACGTCAGTGGAGATATCACATCAATCCA |
| | | CTTGCTTTGAAGACGTGGTTGGAACGTCTTCTTT |
| | | TTCCACGATGCTCCTCGTGGGTGGGGGTCCATC |
| | | TTTGGGACCACTGTCGGCAGAGGCATCTTCAAC |
| | | GATGGCCTTTCCTTTATCGCAATGATGGCATTTG |
| | | TAGGAGCCACCTTCCTTTTCCACTATCTTCACAA |
| | | TAAAGTGACAGATAGCTGGGCAATGGAATCCGA |
| | | GGAGGTTTCCGGATATTACCCTTTGTTGAAAAG |
| | | TCTCAATTGCCCTTTGGTCTTCTGAGACTGTATC |
| | | TTTGATATTTTTGGAGTAGACAAGTGTGTCGTGC |
| | | TCCACCATGTTATCACATCAATCCACTTGCTTTG |
| | | AAGACGTGGTTGGAACGTCTTCTTTTTCCACGAT |
| | | GCTCCTCGTGGGGGGGGTCCATCTTTGGGACC |
| | | ACTGTCGGCAGAGGCATCTTCAACGATGGCCTT |
| | | TCCTTTATCGCAATGATGGCATTTGTAGGAGCC |
| | | ACCTTCCTTTTCCACTATCTTCACAATAAAGTGA |
| | | CAGATAGCTGGGCAATGGAATCCGAGGAGGTTT |
| | | CCGGATATTACCCTTTGTTGAAAAGTCTCAATTG |
| | | CCCTTTGGTCTTCTGAGACTGTATCTTTGATATT |
| | | TTTGGAGTAGACAAGTGTGTCGTGCTCCACCAT |
| | | GTTGACCTGCAGACTAGTCACTATTTTCAGAAG |
| | | AAGTTCCCAATAGTAGTCCAAAATTTTTGTAACG |
| | | AAGGGAGCATAATAGTTACATGCAAAGGAAAAC |
| | | TGCCATTCTTTAGAGGGGATGCTTGTTTAAGAA |
| | | CAAAAAATATATCACTTTCTTTTGTTCCAAGTCA |
| | | TTGCGTATTTTTTTAAAAATATTTGTTCCTTCGT |
| | | ATATTTCGAGCTTCAATCACTTTATGGTTCTTTG |
| | | TATTCTGGCTTTGCTGTAAATCGTAGCTAACCTT |
| | | CTTCCTAGCAGAAATTATTAATACTTGGGATATT |
| | | TTTTTAGAATCAAGTAAATTACATATTACCACCA |
| | | CATCGAGCTGCTTTTAAATTCATATTACAGCCAT |
| | | ATAGGCTTGATTCATTTTGCAAAATTTCCAGGAT |
| | | ATTGACAACGTTAACTTAATAATATCTTGAAATA |
| | | TTAAAGCTATTATGATTAGGGGTGCAAATGGAC |
| | | CGAGTTGGTGGTGTAAATATATGAGCTCCCATT |
| | | TTATTTATTGCTCCCATTTTATTTATTTTAGTTTG |
| | | TGTGACAGATGAACATTATTAGGAGGAAAGGTA |
| | | TAAGCAGGGTTTAACTGTCACAGGGAAGGTGGT |
| | | TTTGGGAGTCTAAAGCTTCAATGATACTTGTGA |
| | | GCAAGAGCATTTGCTACGCCGGCAACAACCTTC |
| | | TGAAATGCAGCCTGCACATTAGGGTTGAAATCA |
| | | TGACCAAGTCTACGGGCTAGGACTACAACAATG |
| | | ACGTTGCCGAGTAGGCGAAAATTTTCAGGGTCG |
| | | ACGTGAAGTTGATCACAATGCAACTCAGATAAC |
| | | TTCGCAAAAGTACCTTTCAGGTTGTCCAAATGCT |
| | | TCAGCCCATCAGAGAATGATTGCAATACTTTCTT |
| | | TCCATGAGCTTTCACTTTTGGATTTCCCATAACA |
| | | GCATCTGCATTGGACAAATCACCAAAGCTCTCA |
| | | AAGAATCTTTGTGTCCAGGGGTAAACCACAAGG |
| | | AGCCTCCCAAGTGCCTCTCCACCAACTTCATCAA |
| | | CATTAACTTTTCCCCACAACCCTAACACTGCTTC |
| | | CTTTTCCTCAGCACTGAGGTGCACACTTCCTCCA |
| | | CCTCCAGATCCTCCACCTCCTGATCCTCCACCTC |
| | | CTGATCCTCCACCTCCACTCCTATATTTGCTGGT |
| | | GAGTACAGTTGAGACGTTGGCGAGGAACTTGTC |
| | | CAAACTGGCATGCACAGAAGGATTAAAATCATC |
| | | TGGATGGTGGGCGGCTAAAGTGACTAACAAACA |
| | | ATGGGACAGCAGCTTGAAATTTACCGGATCAAC |
| | | TCTCAATTTGTGTGCATGAAGATCAGATAATGCA |
| | | GAAAGTGCGCCGGGAAGGTCATCCAAGTGCCCA |
| | | ACAGCTTTTGTCAAAGCATCAGCCACCTTCTGCC |
| | | CATGTGCCTTCACTTGATCACTTCCATGTGAGAG |
| | | GTTGAAATGAGGAAAGTAAGTCTTTGTCGTTGG |
| | | AAAGCCAAGAAACATACGCTCAAGAGCTTCAGC |
| | | ACCGTGCGCTCCAGCTTGACCACCAACTTTTCC |
| | | CCAAGCAGCCTTAACATTTGCTTTGTCTGCTGCC |
| | | GATAGAACCATGGATCCGGCTATCGTTCGTAAA |
| | | TGGTGAAAATTTTCAGAAAATTGCTTTTGCTTTA |
| | | AAAGAAATGATTTAAATTGCTGCAATAGAAGTA |
| | | GAATGCTTGATTGCTTGAGATTCGTTTGTTTTGT |
| | | ATATGTTGTGTTGAGAATTGTTGTGTTAGAGAG |
| | | CTACAATATATAGAGAAAGATCACCGTTTGGCT |
| | | AGCTTTAAGTGGCTAAGTGTGAACGGGATGAGA |
| | | TTCTCTTTTCTTTTGGCCGTGTTGGTCACAAGAT |
| | | TGGACGTCGACGTGTTTGAGGCTTGTGAAATTT |
| | | CCAGAAGCAAAGAAAGGATAAACTTGAGTTAAC |
| | | ATGCAATGGGGAGAAAAACAAAGGGTTGGTGAG |
| | | TGTGAGGGGTACTTGGCAGCTGAGAACAATGCT |
| | | TTAGTTGCATGCCATGCAGATTTCATCCGTTTGA |
| | | AACTCTGTAAAGGACGTGTGGATCGAGTTCGCG |
| | | TGAGCAGCTAGCTTGGCTTGGATTTTTGTCTTGC |
| | | TATCCCTCTACTACATTAATTTCCCTACATGCAA |
| | | CTGTTTCATGACATTTTCGTGTATCTCCCAACTG |
| | | GCTAGCTCATATTAACTAAGGAAAATAGAATTCT |
| | | AATGGAAAATTTAAATAATAGACTTTCATATTTT |
| | | ATTTGTCCCCCTCAAATTTTTTACATTTCATTTTT |
| | | TCGATTTTATTCTATTTATTTTTTTATTCTTTTTT |
| | | TAAAAATTGGCATTCTAAACCCTATATATTATAT |
| | | GAAATAATATATTTTTTAACAAACTCTTTATTAT |
| | | TCATCAAAATTATAAATGCATGAATGAAGTATTA |
| | | TATAAGAAGTGAAACTTATAAAATTATGTAATTT |
| | | ATAATAAGTTTCAAGTACGCGTGTTCACCAACAA |
| | | AATCACGTGAAAAATTGAACAAAAGACGCAAAA |
| | | GCAAGACCAAGTAGCCTCCTGAGTGATGCGCTT |
| | | CAACTGTTGCAAACACTAACCTAAACATAGACG |
| | | GCTTCTAGGGTGCGCAAAGTTGAAATGTGAGGC |
| | | ACGGTACACAAGTTTTTTTAGGACCGTTGGATAT |
| | | AACACTTAATTAGTTAACGGTGCAAATCTCCAAG |
| | | ATTTTAGAAGTGGAAAAAGTATTGAATAAAAAA |
| | | GTATCGCATTTACTGTAGAGCAAACTCTTATTTT |
| | | AATATTGTTGGGTCACGTGGGTGTGGGCTTCTC |
| | | GACTCCACCGGATGATGTTTTCAATTTTGATTTC |
| | | TTTTTTTTCCTAAAGGTTTTCCATTTACATTTATT |
| | | TTGGTTAGAAAAAAGAATAAGGCTAAATGTCTA |
| | | AACCAACTTGCATTCGAACCTAGAACTAAAATAA |
| | | TCTACCACCACGCCATCATCAACTTTATACTTTT |
| | | GAAAAGTATTTATAACAATATATATATTTTATCA |
| | | AACATGCTTAATTGCCTTAAAATAAAATTTATAA |
| | | ATTAGTTGGTAATATTTTAACAATATTAACGGAT |
| | | TTCTGGTAAAAAAAAATAAAATCATGAATTTCTA |
| | | AAATTTTAAAATCAAATTTTTTAATGTATAGATT |
| | | ATTTTATCTAGAGAATTCGTAATCATGTCATAGC |
| | | TGTTTCCTGTGTGAAATTGTTATCCGCTCACAAT |
| | | TCCACACAACATACGAGCCGGAAGCATAAAGTG |
| | | TAAAGCCTGGGGTGCCTAATGAGTGAGCTAACT |
| | | CACATTAATTGCGTTGCGCTCACTGCCCGCTTTC |
| | | CAGTCGGGAAACCTGTCGTGCCAGCTGCATTAA |
| | | TGAATCGGCCAACGCGCGGGGAGAGGCGGTTT |
| | | GCGTATTGGAGCTTGAGCTTGGATCAGATTGTC |
| | | GTTTCCCGCCTTCAGTTTAAACTATCAGTGTTTG |
| | | ACAGGATATATTGGCGGGTAAAC |
| | | 3′ |
| SEQ ID | 35S + TEV + | 5′ |
| NO: 5 | myoglobincDNA + | aataatGCGGCCGCATGGTGGAGCACGACACTCTC |
| | NOS | GTCTACTCCAAGAATATCAAAGATACAGTCTCA |
| | | GAAGACCAAAGGGCTATTGAGACTTTTCAACAA |
| | | AGGGTAATATCGGGAAACCTCCTCGGATTCCAT |
| | | TGCCCAGCTATCTGTCACTTCATCAAAAGGACA |
| | | GTAGAAAAGGAAGGTGGCACCTACAAATGCCAT |
| | | CATTGCGATAAAGGAAAGGCTATCGTTCAAGAT |
| | | GCCTCTGCCGACAGTGGTCCCAAAGATGGACCC |
| | | CCACCCACGAGGAGCATCGTGGAAAAAGAAGAC |
| | | GTTCCAACCACGTCTTCAAAGCAAGTGGATTGA |
| | | TGTGATAACATGGTGGAGCACGACACTCTCGTC |
| | | TACTCCAAGAATATCAAAGATACAGTCTCAGAA |
| | | GACCAAAGGGCTATTGAGACTTTTCAACAAAGG |
| | | GTAATATCGGGAAACCTCCTCGGATTCCATTGC |
| | | CCAGCTATCTGTCACTTCATCAAAAGGACAGTA |
| | | GAAAAGGAAGGTGGCACCTACAAATGCCATCAT |
| | | TGCGATAAAGGAAAGGCTATCGTTCAAGATGCC |
| | | TCTGCCGACAGTGGTCCCAAAGATGGACCCCCA |
| | | CCCACGAGGAGCATCGTGGAAAAAGAAGACGTT |
| | | CCAACCACGTCTTCAAAGCAAGTGGATTGATGT |
| | | GATATCTCCACTGACGTAAGGGATGACGCACAA |
| | | TCCCACTATCCTTCGCAAGACCTTCCTCTATATA |
| | | AGGAAGTTCATTTCATTTGGAGAGGACACGCTG |
| | | AAATCACCAGTCTCTCTCTACAAATCTATCTCTG |
| | | AATTAATTCTCAACACAACATATACAAAACAAAC |
| | | GAATCTCAAGCAATCAAGCATTCTACTTCTATTG |
| | | CAGCAATTTAAATCATTTCTTTTAAAGCAAAAGC |
| | | AATTTTCTGAAAATTTTCACCATTTACGAACGAT |
| | | AGATGGGGCTATCAGATGGTGAATGGCAACTTG |
| | | TATTGAATGTTTGGGGAAAAGTTGAAGCTGATG |
| | | TTGCTGGACATGGTCAAGAAGTGTTAATAAGAC |
| | | TCTTCAAAGGCCACCCTGAAACATTAGAGAAGT |
| | | TTGACAAATTCAAGCACCTAAAATCTGAAGATG |
| | | AAATGAAGGCCTCCGAGGACTTGAAGAAGCATG |
| | | GAAACACTGTCCTGACTGCACTCGGCGGGATCC |
| | | TCAAAAAGAAAGGTCATCATGAAGCGGAGTTGA |
| | | CACCATTGGCTCAGTCTCATGCTACCAAACACA |
| | | AGATTCCTGTGAAGTATCTTGAGTTTATTAGTGA |
| | | GGCCATAATTCAGGTTTTGCAATCAAAACATCCC |
| | | GGTGATTTTGGTGCAGATGCTCAAGGAGCAATG |
| | | AGCAAAGCACTGGAGCTTTTCAGGAATGATATG |
| | | GCAGCCAAGTACAAGGAACTTGGATTTCAGGGG |
| | | TGAGATCGTTCAAACATTTGGCAATAAAGTTTCT |
| | | TAAGATTGAATCCTGTTGCCGGTCTTGCGATGA |
| | | TTATCATATAATTTCTGTTGAATTACGTTAAGCA |
| | | TGTAATAATTAACATGTAATGCATGACGTTATTT |
| | | ATGAGATGGGTTTTTATGATTAGAGTCCCGCAA |
| | | TTATACATTTAATACGCGATAGAAAACAAAATAT |
| | | AGCGCGCAAACTAGGATAAATTATCGCGCGCGG |
| | | TGTCATCTATGTTACTAGATCGGCGGCCGCttatta |
| | | 3′ |
| SEQ ID | p7S + TEV + | 5′ |
| NO: 6 | myoglobincDNA + | aaatttGCGGCCGCGGTACTTGGCAGCTGAGAACA |
| | arc5 + Rb7MAR | ATGCTTTAGTTGCATGCCATGCAGATTTCATCCG |
| | | TTTGAAACTCTGTAAAGGACGTGTGGATCGAGT |
| | | TCGCGTGAGCAGCTAGCTTGGCTTGGATTTTTG |
| | | TCTTGCTATCCCTCTACTACATTAATTTCCCTAC |
| | | ATGCAACTGTTTCATGACATTTTCGTGTATCTCC |
| | | CAACTGGCTAGCTCATATTAACTAAGGAAAATA |
| | | GAATTCTAATGGAAAATTTAAATAATAGACTTTC |
| | | ATATTTTATTTGTCCCCCTCAAATTTTTTACATTT |
| | | CATTTTTTCGATTTTATTCTATTTATTTTTTTATT |
| | | CTTTTTTTAAAAATTGGCATTCTAAACCCTATAT |
| | | ATTATATGAAATAATATATTTTTTAACAAACTCT |
| | | TTATTATTCATCAAAATTATAAATGCATGAATGA |
| | | AGTATTATATAAGAAGTGAAACTTATAAAATTAT |
| | | GTAATTTATAATAAGTTTCAAGTACGCGTGTTCA |
| | | CCAACAAAATCACGTGAAAAATTGAACAAAAGA |
| | | CGCAAAAGCAAGACCAAGTAGCCTCCTGAGTGA |
| | | TGCGCTTCAACTGTTGCAAACACTAACCTAAACA |
| | | TAGACGGCTTCTAGGGTGCGCAAAGTTGAAATG |
| | | TGAGGCACGGTACACAAGTTTTTTTAGGACCGT |
| | | TGGATATAACACTTAATTAGTTAACGGTGCAAAT |
| | | CTCCAAGATTTTAGAAGTGGAAAAAGTATTGAA |
| | | TAAAAAAGTATCGCATTTACTGTAGAGCAAACTC |
| | | TTATTTTAATATTGTTGGGTCACGTGGGTGTGG |
| | | GCTTCTCGACTCCACCGGATGATGTTTTCAATTT |
| | | TGATTTCTTTTTTTTGCTAAAGGTTTTCCATTTA |
| | | CATTTATTTTGGTTAGAAAAAAGAATAAGGCTAA |
| | | ATGCCTAAACCAACTTGCATTCGAACCTAGAACT |
| | | AAAATAATCTACCACCACGCCATCATCAACTTTA |
| | | TACTTTTGAAAAGTATTTATAACAATATATATAT |
| | | TTTATCAAACATGCTTAATTGCCTTAAAATAAAA |
| | | TTTATAAATTAGTTGGTAATATTTTAACAATATT |
| | | AACAAATTTCTGGTAAAAAAATTAAAATCATGAA |
| | | TTTCTAAAATTTTAAAATCAAATTTTTTAATGTAT |
| | | AGGTTATTTTAATTTAATTTATATTGTTAAAAAT |
| | | GGGTTATTACAATTTAATTAATCTCTAATTAAAA |
| | | TATATATGAGGATATTAGTTTTATTCTAATACTA |
| | | TTGTAATTCCCATTCATATGAATATATATATATG |
| | | AGATTGTTTGATAAAATAATCTCAATAAATGTTT |
| | | CAACTATGCAAAGCATAATGAGCATCTAACTTAT |
| | | TTACAATTTACAATAATAAATCATGATCAAAACA |
| | | AACATCAACACTAACTCGTTATTAGTATGTTATC |
| | | AACAAATGTAAAGGTGAAGCTACTCTAGCTTAA |
| | | GTCAAAGTTAAATTATTCTGATCAGAAACTCTTT |
| | | GAAAATATACCTAATAAAACTCAAGAAAATACAC |
| | | ACCTAATATAAACATATGTTGAACATCTTTATAC |
| | | ATGTGCATCTTCAAACCCCTATTCATTCCATCTA |
| | | TTGTGTCTCCAAGAAGGAGATCCAACCTATAAG |
| | | TCATTTCCTCCCATGTACGTCATGTATAGGATCA |
| | | TTACAGTCACAGCCACATGTGTTATATGCAAGG |
| | | ATTTAATTAGTAGTGTAAAAACTTGAGTGGAATC |
| | | CTTCAAGGATTAGAAAGAGAAACTAAGGTAAAT |
| | | TAAGAGATTTACTGAATCAATATAAATCTTTTGT |
| | | TTTTACTCGAGCTTCTATTATACTTGTTTTATCTT |
| | | AGTTGCTTCTATCTTACTTTCGATGTTTAAATTT |
| | | TGAGAAAAAAATCCTTTTGTGAAAAACCTTTTTA |
| | | AAAAGTTGTTTGACGCTACCGTACAGAGCATCC |
| | | ATTTTTAATTTGTGGTCAAATTTGCTTTGTGAAA |
| | | ATCTTCATCTTACAAAAAAAAAACCTTAATTTAA |
| | | ATCTCATCTAACATATTGAATTAATTCTCAACAC |
| | | AACATATACAAAACAAACGAATCTCAAGCAATC |
| | | AAGCATTCTACTTCTATTGCAGCAATTTAAATCA |
| | | TTTCTTTTAAAGCAAAAGCAATTTTCTGAAAATT |
| | | TTCACCATTTACGAACGATAGATGGGGCTATCA |
| | | GATGGTGAATGGCAACTTGTATTGAATGTTTGG |
| | | GGAAAAGTTGAAGCTGATGTTGCTGGACATGGT |
| | | CAAGAAGTGTTAATAAGACTCTTCAAAGGCCAC |
| | | CCTGAAACATTAGAGAAGTTTGACAAATTCAAG |
| | | CACCTAAAATCTGAAGATGAAATGAAGGCCTCC |
| | | GAGGACTTGAAGAAGCATGGAAACACTGTCCTG |
| | | ACTGCACTCGGCGGGATCCTCAAAAAGAAAGGT |
| | | CATCATGAAGCGGAGTTGACACCATTGGCTCAG |
| | | TCTCATGCTACCAAACACAAGATTCCTGTGAAGT |
| | | ATCTTGAGTTTATTAGTGAGGCCATAATTCAGGT |
| | | TTTGCAATCAAAACATCCCGGTGATTTTGGTGCA |
| | | GATGCTCAAGGAGCAATGAGCAAAGCACTGGAG |
| | | CTTTTCAGGAATGATATGGCAGCCAAGTACAAG |
| | | GAACTTGGATTTCAGGGGTGATAGACTCCCAAA |
| | | ACCACCTTCCCTGTGACAGTTAAACCCTGCTTAT |
| | | ACCTTTCCTCCTAATAATGTTCATCTGTCACACA |
| | | AACTAAAATAAATAAAATGGGAGCAATAAATAA |
| | | AATGGGAGCTCATATATTTACACCACCAACTCG |
| | | GTCCATTTGCACCCCTAATCATAATAGCTTTAAT |
| | | ATTTCAAGATATTATTAAGTTAACGTTGTCAATA |
| | | TCCTGGAAATTTTGCAAAATGAATCAAGCCTATA |
| | | TGGCTGTAATATGAATTTAAAAGCAGCTCGATG |
| | | TGGTGGTAATATGTAATTTACTTGATTCTAAAAA |
| | | AATATCCCAAGTATTAATAATTTCTGCTAGGAAG |
| | | AAGGTTAGCTACGATTTACAGCAAAGCCAGAAT |
| | | ACAAAGAACCATAAAGTGATTGAAGCTCGAAAT |
| | | ATACGAAGGAACAAATATTTTTAAAAAAATACGC |
| | | AATGACTTGGAACAAAAGAAAGTGATATATTTTT |
| | | TGTTCTTAAACAAGCATCCCCTCTAAAGAATGGC |
| | | AGTTTTCCTTTGCATGTAACTATTATGCTCCCTT |
| | | CGTTACAAAAATTTTGGACTACTATTGGGAACTT |
| | | CTTCTGAAAATAGTGGCGGCCGCttaata |
| | | 3′ |
| SEQ ID | PPhas + | 5′ |
| NO: 7 | myoglobincDNA + | aaatttGCGGCCGCCATTGTACTCCCAGTATCATTA |
| | arc5 + Rb7MAR | TAGTGAAAGTTTTGGCTCTCTCGCCGGTGGTTTT |
| | | TTACCTCTATTTAAAGGGGTTTTCCACCTAAAAA |
| | | TTCTGGTATCATTCTCACTTTACTTGTTACTTTA |
| | | ATTTCTCATAATCTTTGGTTGAAATTATCACGCT |
| | | TCCGCACACGATATCCCTACAAATTTATTATTTG |
| | | TTAAACATTTTCAAACCGCATAAAATTTTATGAA |
| | | GTCCCGTCTATCTTTAATGTAGTCTAACATTTTC |
| | | ATATTGAAATATATAATTTACTTAATTTTAGCGT |
| | | TGGTAGAAAGCATAAAGATTTATTCTTATTCTTC |
| | | TTCATATAAATGTTTAATATACAATATAAACAAA |
| | | TTCTTTACCTTAAGAAGGATTTCCCATTTTATAT |
| | | TTTAAAAATATATTTATCAAATATTTTTCAACCA |
| | | CGTAAATCTCATAATAATAAGTTGTTTCAAAAGT |
| | | AATAAAATTTAACTCCATAATTTTTTTATTCGAC |
| | | TGATCTTAAAGCAACACCCAGTGACACAACTAG |
| | | CCATTTTTTTCTTTGAATAAAAAAATCCAATTAT |
| | | CATTGTATTTTTTTTATACAATGAAAATTTCACC |
| | | AAACAATCATTTGTGGTATTTCTGAAGCAAGTCA |
| | | TGTTATGCAAAATTCTATAATTCCCATTTGACAC |
| | | TACGGAAGTAACTGAAGATCTGCTTTTACATGC |
| | | GAGACACATCTTCTAAAGTAATTTTAATAATAGT |
| | | TACTATATTCAAGATTTCATATATCAAATACTCA |
| | | ATATTACTTCTAAAAAATTAATTAGATATAATTA |
| | | AAATATTACTTTTTTAATTTTAAGTTTAATTGTTG |
| | | AATTTGTGACTATTGATTTATTATTCTACTATGT |
| | | TTAAATTGTTTTATAGATAGTTTAAAGTAAATAT |
| | | AAGTAATGTAGTAGAGTGTTAGAGTGTTACCCT |
| | | AAACCATAAACTATAACATTTATGGTGGACTAAT |
| | | TTTCATATATTTCTTATTGCTTTTACCTTTTCTTG |
| | | GTATGTAAGTCCGTAACTAGAATTACAGTGGGT |
| | | TGCCATGGCACTCTGTGGTCTTTTGGTTCATGCA |
| | | TGGGTCTTGCGCAAGAAAAAGACAAAGAACAAA |
| | | GAAAAAAGACAAAACAGAGAGACAAAACGCAAT |
| | | CACACAACCAACTCAAATTAGTCACTGGCTGAT |
| | | CAAGATCGCCGCGTCCATGTATGTCTAAATGCC |
| | | ATGCAAAGCAACACGTGCTTAACATGCACTTTA |
| | | AATGGCTCACCCATCTCAACCCACACACAAACA |
| | | CATTGCCTTTTTCTTCATCATCACCACAACCACC |
| | | TGTATATATTCATTCTCTTCCGCCACCTCAATTT |
| | | CTTCACTTCAACACACGTCAACCTGCATATGCGT |
| | | GTCATCCCATGCCCAAATCTCCATGCATGTTCCA |
| | | ACCACCTTCTCTCTTATATAATACCTATAAATAC |
| | | CTCTAATATCACTCACTTCTTTCATCATCCATCC |
| | | ATCCAGAGTACTACTACTCTACTACTATAATACC |
| | | CCAACCCAACTCATATTCAATACTACTCTACTAT |
| | | GGGGCTATCAGATGGTGAATGGCAACTTGTATT |
| | | GAATGTTTGGGGAAAAGTTGAAGCTGATGTTGC |
| | | TGGACATGGTCAAGAAGTGTTAATAAGACTCTT |
| | | CAAAGGCCACCCTGAAACATTAGAGAAGTTTGA |
| | | CAAATTCAAGCACCTAAAATCTGAAGATGAAAT |
| | | GAAGGCCTCCGAGGACTTGAAGAAGCATGGAAA |
| | | CACTGTCCTGACTGCACTCGGCGGGATCCTCAA |
| | | AAAGAAAGGTCATCATGAAGCGGAGTTGACACC |
| | | ATTGGCTCAGTCTCATGCTACCAAACACAAGATT |
| | | CCTGTGAAGTATCTTGAGTTTATTAGTGAGGCC |
| | | ATAATTCAGGTTTTGCAATCAAAACATCCCGGTG |
| | | ATTTTGGTGCAGATGCTCAAGGAGCAATGAGCA |
| | | AAGCACTGGAGCTTTTCAGGAATGATATGGCAG |
| | | CCAAGTACAAGGAACTTGGATTTCAGGGGTGAT |
| | | AGACTCCCAAAACCACCTTCCCTGTGACAGTTA |
| | | AACCCTGCTTATACCTTTCCTCCTAATAATGTTC |
| | | ATCTGTCACACAAACTAAAATAAATAAAATGGG |
| | | AGCAATAAATAAAATGGGAGCTCATATATTTACA |
| | | CCACCAACTCGGTCCATTTGCACCCCTAATCATA |
| | | ATAGCTTTAATATTTCAAGATATTATTAAGTTAA |
| | | CGTTGTCAATATCCTGGAAATTTTGCAAAATGAA |
| | | TCAAGCCTATATGGCTGTAATATGAATTTAAAAG |
| | | CAGCTCGATGTGGTGGTAATATGTAATTTACTTG |
| | | ATTCTAAAAAAATATCCCAAGTATTAATAATTTC |
| | | TGCTAGGAAGAAGGTTAGCTACGATTTACAGCA |
| | | AAGCCAGAATACAAAGAACCATAAAGTGATTGA |
| | | AGCTCGAAATATACGAAGGAACAAATATTTTTAA |
| | | AAAAATACGCAATGACTTGGAACAAAAGAAAGT |
| | | GATATATTTTTTGTTCTTAAACAAGCATCCCCTC |
| | | TAAAGAATGGCAGTTTTCCTTTGCATGTAACTAT |
| | | TATGCTCCCTTCGTTACAAAAATTTTGGACTACT |
| | | ATTGGGAACTTCTTCTGAAAATAGTGGCGGCCG |
| | | Cttaaat |
| | | 3′ |
| SEQ ID | 2x 35S promoter | 5′ |
| NO: 8 | | ATGGTGGAGCACGACACTCTCGTCTACTCCAAG |
| | | AATATCAAAGATACAGTCTCAGAAGACCAAAGG |
| | | GCTATTGAGACTTTTCAACAAAGGGTAATATCG |
| | | GGAAACCTCCTCGGATTCCATTGCCCAGCTATC |
| | | TGTCACTTCATCAAAAGGACAGTAGAAAAGGAA |
| | | GGTGGCACCTACAAATGCCATCATTGCGATAAA |
| | | GGAAAGGCTATCGTTCAAGATGCCTCTGCCGAC |
| | | AGTGGTCCCAAAGATGGACCCCCACCCACGAGG |
| | | AGCATCGTGGAAAAAGAAGACGTTCCAACCACG |
| | | TCTTCAAAGCAAGTGGATTGATGTGATAACATG |
| | | GTGGAGCACGACACTCTCGTCTACTCCAAGAAT |
| | | ATCAAAGATACAGTCTCAGAAGACCAAAGGGCT |
| | | ATTGAGACTTTTCAACAAAGGGTAATATCGGGA |
| | | AACCTCCTCGGATTCCATTGCCCAGCTATCTGTC |
| | | ACTTCATCAAAAGGACAGTAGAAAAGGAAGGTG |
| | | GCACCTACAAATGCCATCATTGCGATAAAGGAA |
| | | AGGCTATCGTTCAAGATGCCTCTGCCGACAGTG |
| | | GTCCCAAAGATGGACCCCCACCCACGAGGAGCA |
| | | TCGTGGAAAAAGAAGACGTTCCAACCACGTCTT |
| | | CAAAGCAAGTGGATTGATGTGATATCTCCACTG |
| | | ACGTAAGGGATGACGCACAATCCCACTATCCTT |
| | | CGCAAGACCTTCCTCTATATAAGGAAGTTCATTT |
| | | CATTTGGAGAGGACACGCTGAAATCACCAGTCT |
| | | CTCTCTACAAATCTATCTCT |
| | | 3′ |
| SEQ ID | TEV | 5′ |
| NO: 9 | | GAATTAATTCTCAACACAACATATACAAAACAAA |
| | | CGAATCTCAAGCAATCAAGCATTCTACTTCTATT |
| | | GCAGCAATTTAAATCATTTCTTTTAAAGCAAAAG |
| | | CAATTTTCTGAAAATTTTCACCATTTACGAACGA |
| | | TAG |
| | | 3′ |
| SEQ ID | NOS terminator | 5′ |
| NO: 10 | | GATCGTTCAAACATTTGGCAATAAAGTTTCTTAA |
| | | GATTGAATCCTGTTGCCGGTCTTGCGATGATTAT |
| | | CATATAATTTCTGTTGAATTACGTTAAGCATGTA |
| | | ATAATTAACATGTAATGCATGACGTTATTTATGA |
| | | GATGGGTTTTTATGATTAGAGTCCCGCAATTATA |
| | | CATTTAATACGCGATAGAAAACAAAATATAGCG |
| | | CGCAAACTAGGATAAATTATCGCGCGCGGTGTC |
| | | ATCTATGTTACTAGATC |
| | | 3′ |
| SEQ ID | 7S promoter | 5′ |
| NO: 11 | | GGTACTTGGCAGCTGAGAACAATGCTTTAGTTG |
| | | CATGCCATGCAGATTTCATCCGTTTGAAACTCTG |
| | | TAAAGGACGTGTGGATCGAGTTCGCGTGAGCAG |
| | | CTAGCTTGGCTTGGATTTTTGTCTTGCTATCCCT |
| | | CTACTACATTAATTTCCCTACATGCAACTGTTTC |
| | | ATGACATTTTCGTGTATCTCCCAACTGGCTAGCT |
| | | CATATTAACTAAGGAAAATAGAATTCTAATGGAA |
| | | AATTTAAATAATAGACTTTCATATTTTATTTGTC |
| | | CCCCTCAAATTTTTTACATTTCATTTTTTCGATTT |
| | | TATTCTATTTATTTTTTTATTCTTTTTTTAAAAAT |
| | | TGGCATTCTAAACCCTATATATTATATGAAATAA |
| | | TATATTTTTTAACAAACTCTTTATTATTCATCAAA |
| | | ATTATAAATGCATGAATGAAGTATTATATAAGAA |
| | | GTGAAACTTATAAAATTATGTAATTTATAATAAG |
| | | TTTCAAGTACGCGTGTTCACCAACAAAATCACGT |
| | | GAAAAATTGAACAAAAGACGCAAAAGCAAGACC |
| | | AAGTAGCCTCCTGAGTGATGCGCTTCAACTGTT |
| | | GCAAACACTAACCTAAACATAGACGGCTTCTAG |
| | | GGTGCGCAAAGTTGAAATGTGAGGCACGGTACA |
| | | CAAGTTTTTTTAGGACCGTTGGATATAACACTTA |
| | | ATTAGTTAACGGTGCAAATCTCCAAGATTTTAGA |
| | | AGTGGAAAAAGTATTGAATAAAAAAGTATCGCA |
| | | TTTACTGTAGAGCAAACTCTTATTTTAATATTGT |
| | | TGGGTCACGTGGGTGTGGGCTTCTCGACTCCAC |
| | | CGGATGATGTTTTCAATTTTGATTTCTTTTTTTT |
| | | GCTAAAGGTTTTCCATTTACATTTATTTTGGTTA |
| | | GAAAAAAGAATAAGGCTAAATGCCTAAACCAAC |
| | | TTGCATTCGAACCTAGAACTAAAATAATCTACCA |
| | | CCACGCCATCATCAACTTTATACTTTTGAAAAGT |
| | | ATTTATAACAATATATATATTTTATCAAACATGC |
| | | TTAATTGCCTTAAAATAAAATTTATAAATTAGTT |
| | | GGTAATATTTTAACAATATTAACAAATTTCTGGT |
| | | AAAAAAATTAAAATCATGAATTTCTAAAATTTTA |
| | | AAATCAAATTTTTTAATGTATAGGTTATTTTAAT |
| | | TTAATTTATATTGTTAAAAATGGGTTATTACAAT |
| | | TTAATTAATCTCTAATTAAAATATATATGAGGAT |
| | | ATTAGTTTTATTCTAATACTATTGTAATTCCCAT |
| | | TCATATGAATATATATATATGAGATTGTTTGATA |
| | | AAATAATCTCAATAAATGTTTCAACTATGCAAAG |
| | | CATAATGAGCATCTAACTTATTTACAATTTACAA |
| | | TAATAAATCATGATCAAAACAAACATCAACACTA |
| | | ACTCGTTATTAGTATGTTATCAACAAATGTAAAG |
| | | GTGAAGCTACTCTAGCTTAAGTCAAAGTTAAATT |
| | | ATTCTGATCAGAAACTCTTTGAAAATATACCTAA |
| | | TAAAACTCAAGAAAATACACACCTAATATAAACA |
| | | TATGTTGAACATCTTTATACATGTGCATCTTCAA |
| | | ACCCCTATTCATTCCATCTATTGTGTCTCCAAGA |
| | | AGGAGATCCAACCTATAAGTCATTTCCTCCCATG |
| | | TACGTCATGTATAGGATCATTACAGTCACAGCC |
| | | ACATGTGTTATATGCAAGGATTTAATTAGTAGTG |
| | | TAAAAACTTGAGTGGAATCCTTCAAGGATTAGA |
| | | AAGAGAAACTAAGGTAAATTAAGAGATTTACTG |
| | | AATCAATATAAATCTTTTGTTTTTACTCGAGCTT |
| | | CTATTATACTTGTTTTATCTTAGTTGCTTCTATCT |
| | | TACTTTCGATGTTTAAATTTTGAGAAAAAAATCC |
| | | TTTTGTGAAAAACCTTTTTAAAAAGTTGTTTGAC |
| | | GCTACCGTACAGAGCATCCATTTTTAATTTGTGG |
| | | TCAAATTTGCTTTGTGAAAATCTTCATCTTACAA |
| | | AAAAAAAACCTTAATTTAAATCTCATCTAACATA |
| | | TT |
| | | 3′ |
| SEQ ID | Arc5 Terminator | 5′ |
| NO: 12 | | TAGACTCCCAAAACCACCTTCCCTGTGACAGTTA |
| | | AACCCTGCTTATACCTTTCCTCCTAATAATGTTC |
| | | ATCTGTCACACAAACTAAAATAAATAAAATGGG |
| | | AGCAATAAATAAAATGGGAGCTCATATATTTACA |
| | | CCA |
| | | 3′ |
| SEQ ID | Rb7 Mar | 5′ |
| NO: 13 | | CCAACTCGGTCCATTTGCACCCCTAATCATAATA |
| | | GCTTTAATATTTCAAGATATTATTAAGTTAACGT |
| | | TGTCAATATCCTGGAAATTTTGCAAAATGAATCA |
| | | AGCCTATATGGCTGTAATATGAATTTAAAAGCA |
| | | GCTCGATGTGGTGGTAATATGTAATTTACTTGAT |
| | | TCTAAAAAAATATCCCAAGTATTAATAATTTCTG |
| | | CTAGGAAGAAGGTTAGCTACGATTTACAGCAAA |
| | | GCCAGAATACAAAGAACCATAAAGTGATTGAAG |
| | | CTCGAAATATACGAAGGAACAAATATTTTTAAAA |
| | | AAATACGCAATGACTTGGAACAAAAGAAAGTGA |
| | | TATATTTTTTGTTCTTAAACAAGCATCCCCTCTA |
| | | AAGAATGGCAGTTTTCCTTTGCATGTAACTATTA |
| | | TGCTCCCTTCGTTACAAAAATTTTGGACTACTAT |
| | | TGGGAACTTCTTCTGAAAATAGTG |
| | | 3′ |
| SEQ ID | Phas promoter | 5′ |
| NO: 14 | | CATTGTACTCCCAGTATCATTATAGTGAAAGTTT |
| | | TGGCTCTCTCGCCGGTGGTTTTTTACCTCTATTT |
| | | AAAGGGGTTTTCCACCTAAAAATTCTGGTATCAT |
| | | TCTCACTTTACTTGTTACTTTAATTTCTCATAATC |
| | | TTTGGTTGAAATTATCACGCTTCCGCACACGATA |
| | | TCCCTACAAATTTATTATTTGTTAAACATTTTCA |
| | | AACCGCATAAAATTTTATGAAGTCCCGTCTATCT |
| | | TTAATGTAGTCTAACATTTTCATATTGAAATATA |
| | | TAATTTACTTAATTTTAGCGTTGGTAGAAAGCAT |
| | | AAAGATTTATTCTTATTCTTCTTCATATAAATGT |
| | | TTAATATACAATATAAACAAATTCTTTACCTTAA |
| | | GAAGGATTTCCCATTTTATATTTTAAAAATATAT |
| | | TTATCAAATATTTTTCAACCACGTAAATCTCATA |
| | | ATAATAAGTTGTTTCAAAAGTAATAAAATTTAAC |
| | | TCCATAATTTTTTTATTCGACTGATCTTAAAGCA |
| | | ACACCCAGTGACACAACTAGCCATTTTTTTCTTT |
| | | GAATAAAAAAATCCAATTATCATTGTATTTTTTT |
| | | TATACAATGAAAATTTCACCAAACAATCATTTGT |
| | | GGTATTTCTGAAGCAAGTCATGTTATGCAAAATT |
| | | CTATAATTCCCATTTGACACTACGGAAGTAACTG |
| | | AAGATCTGCTTTTACATGCGAGACACATCTTCTA |
| | | AAGTAATTTTAATAATAGTTACTATATTCAAGAT |
| | | TTCATATATCAAATACTCAATATTACTTCTAAAA |
| | | AATTAATTAGATATAATTAAAATATTACTTTTTT |
| | | AATTTTAAGTTTAATTGTTGAATTTGTGACTATT |
| | | GATTTATTATTCTACTATGTTTAAATTGTTTTATA |
| | | GATAGTTTAAAGTAAATATAAGTAATGTAGTAGA |
| | | GTGTTAGAGTGTTACCCTAAACCATAAACTATAA |
| | | CATTTATGGTGGACTAATTTTCATATATTTCTTA |
| | | TTGCTTTTACCTTTTCTTGGTATGTAAGTCCGTA |
| | | ACTAGAATTACAGTGGGTTGCCATGGCACTCTG |
| | | TGGTCTTTTGGTTCATGCATGGGTCTTGCGCAA |
| | | GAAAAAGACAAAGAACAAAGAAAAAAGACAAAA |
| | | CAGAGAGACAAAACGCAATCACACAACCAACTC |
| | | AAATTAGTCACTGGCTGATCAAGATCGCCGCGT |
| | | CCATGTATGTCTAAATGCCATGCAAAGCAACAC |
| | | GTGCTTAACATGCACTTTAAATGGCTCACCCATC |
| | | TCAACCCACACACAAACACATTGCCTTTTTCTTC |
| | | ATCATCACCACAACCACCTGTATATATTCATTCT |
| | | CTTCCGCCACCTCAATTTCTTCACTTCAACACAC |
| | | GTCAACCTGCATATGCGTGTCATCCCATGCCCA |
| | | AATCTCCATGCATGTTCCAACCACCTTCTCTCTT |
| | | ATATAATACCTATAAATACCTCTAATATCACTCA |
| | | CTTCTTTCATCATCCATCCATCCAGAGTACTACT |
| | | ACTCTACTACTATAATACCCCAACCCAACTCATA |
| | | TTCAATACTACTCTACT |
| | | 3′ |

1-74. (canceled)

75. A transgenic plant comprising a nucleic acid encoding for a hemeprotein, wherein the nucleic acid is operatively linked to:
a phas seed-specific promoter;
an arc5 terminator; and
a Rb7Mar 3′ Matrix Attachment Region;
wherein the hemeprotein is expressed in the plant seeds and comprises about 1% to about 30% of the total soluble protein of the seed.

76. The transgenic plant of claim 75, wherein the phas seed-specific promoter comprises a sequence with at least 70% identity to SEQ ID NO: 14, the arc5 terminator comprises a sequence with at least 70% identity to SEQ ID NO: 12, and the Rb7Mar 3′ Matrix Attachment Region comprises a sequence with at least 70% identity to SEQ ID NO: 13.

77. The transgenic plant of claim 75, wherein the hemeprotein is selected from a plant derived heme protein, a microorganism derived heme protein, or an animal derived heme protein.

78. The transgenic plant of claim 75, wherein the heme protein comprises a heme protein involved in oxygen transport, an enzyme having a heme prosthetic group, or a heme protein involved in the electron transport chain.

79. The transgenic plant of claim 75, wherein the heme protein is selected from: hemoglobin, myoglobin, neuroglobin, cytoglobin, cytochrome P450s, cytochrome c oxidase, ligninases, catalase, peroxidases, cytochrome a, cytochrome b, or cytochrome c.

80. The transgenic plant of claim 75, wherein the heme protein is an animal derived heme protein selected from the group consisting of hemoglobin and myoglobin.

81. The transgenic plant of claim 75, wherein the plant is a legume plant.

82. The transgenic plant of claim 75, wherein the plant is selected from Glycine max, Oryza sativa, Hordeum vulgare, Zea mays, Secale cereale, Avena sativa, Beta vulgaris, Beta vulgaris subspecies vulgaris, Pastinaca sativa, Phaseolus vulgaris, Pisum sativum, Vigna angularis, Vigna radiata, Cicer arietimim, Arachis hypogaea, Lens culinaris, Medicago sativa, Eruca vesicaria, Brassica juncea, Lactuca sativa, Brassica, Solanum tuberosum, Ipomoea batatas, Manihot esculenta, Triticum aestivum and Triticum spelta.

83. A transgenic seed comprising a nucleic acid encoding for a hemeprotein, wherein said nucleic acid is operatively linked to:
a phas seed-specific promoter;
an arc5 terminator; and
a Rb7Mar 3′ Matrix Attachment Region;
wherein said hemeprotein is expressed in the transgenic seed between about 1% to about 30% of the total soluble protein of the transgenic seed.

84. The transgenic seed of claim 83, wherein the phas seed-specific promoter comprises a sequence with at least 70% identity to SEQ ID NO: 14, the arc5 terminator comprises a sequence with at least 70% identity to SEQ ID NO: 12, and the Rb7Mar 3′ Matrix Attachment Region comprises a sequence with at least 70% identity to SEQ ID NO: 13.

85. The transgenic seed of claim 83, wherein said hemeprotein is selected from a plant derived hemeprotein, a microorganism derived hemeprotein, or an animal derived hemeprotein.

86. The transgenic seed of claim 83, wherein said heme protein comprises a hemeprotein involved in oxygen transport, an enzyme having a heme prosthetic group, or a heme protein involved in the electron transport chain.

87. The transgenic seed of claim 83, wherein said heme protein is selected from: hemoglobin, myoglobin, neuroglobin, cytoglobin, cytochrome P450s, cytochrome c oxidase, ligninases, catalase, peroxidases, cytochrome a, cytochrome b, or cytochrome c.

88. The transgenic seed of claim 83, wherein said heme protein is an animal derived heme protein selected from the group consisting of hemoglobin and myoglobin.

89. The transgenic seed of claim 83, wherein the seed is a legume seed.

90. The transgenic seed of claim 83, wherein the seed is selected from Glycine max, Oryza sativa, Hordeum vulgare, Zea mays, Secale cereale, Avena sativa, Beta vulgaris, Beta vulgaris subspecies vulgaris, Pastinaca sativa, Phaseolus vulgaris, Pisum sativum, Vigna angularis, Vigna radiata, Cicer arietimim, Arachis hypogaea, Lens culinaris, Medicago sativa, Eruca vesicaria, Brassica juncea, Lactuca sativa, Brassica, Solanum tuberosum, Ipomoea batatas, Manihot esculenta, Triticum aestivum and Triticum spelta.

91. A food composition comprising the transgenic plant, or plant seeds of claim 75.

92. The food composition of claim 91, comprising a hemeprotein isolated from the transgenic plant.

93. The food composition of claim 91 comprising the seed of the transgenic plant.

94. The food composition of claim 93, wherein the food is texturized.

95. The food composition of claim 93, wherein the food is a meat analog.

96. A method to produce hemeproteins in a plant seed, the method comprises:
transforming a plant with a nucleic acid encoding for a hemeprotein, wherein the nucleic acid is operatively linked to a phas seed-specific promoter, an arc5 terminator and a Rb7Mar 3′ Matrix Attachment Region;
selecting the transformed plants, and
cultivating the transformed plants to produce the hemeproteins;
wherein the hemeprotein comprises about 1% to about 30% the total soluble proteins in the seed.

97. The method of claim 96, wherein the phas seed-specific promoter comprises a sequence with at least 70% identity to SEQ ID NO: 14, the arc5 terminator comprises a sequence with at least 70% identity to SEQ ID NO: 12, and the Rb7Mar 3′ Matrix Attachment Region comprises a sequence with at least 70% identity to SEQ ID NO: 13.

98. The method of claim 96, wherein the plant seed is a legume plant seed.

99. The method of claim 96, wherein the plant seed is selected from Glycine max, Oryza sativa, Hordeum vulgare, Zea mays, Secale cereale, Avena sativa, Beta vulgaris, Beta vulgaris subspecies vulgaris, Pastinaca sativa, Phaseolus vulgaris, Pisum sativum, Vigna angularis, Vigna radiata, Cicer arietinum, Arachis hypogaea, Lens culinaris, Medicago sativa, Eruca vesicaria, Brassica juncea, Lactuca sativa, Brassica, Solanum tuberosum, Ipomoea batatas, Manihot esculenta, Triticum aestivum and Triticum spelta.

100. The method of claim 96, wherein the hemeprotein is selected from a plant derived heme protein, a microorganism derived heme protein, or an animal derived heme protein.

101. The method of claim 96, wherein said hemeprotein comprises a hemeprotein involved in oxygen transport, an enzyme having a heme prosthetic group, or a hemeprotein involved in the electron transport chain.

102. The method of claim 96, wherein said heme protein is selected from: hemoglobin, myoglobin, neuroglobin, cytoglobin, cytochrome P450s, cytochrome c oxidase, ligninases, catalase, peroxidases, cytochrome a, cytochrome b, or cytochrome c.

103. The method of claim 96, wherein said heme protein is an animal derived heme protein selected from the group consisting of hemoglobin and myoglobin.

104. An isolated hemeprotein produced by the method of claim 96.


SEQUENCE LISTING (14 sequences)

SEQ ID NO: 1 - DNA, 930 bp, synthetic construct
  misc_feature 1..930: note = porcine hemoglobin (HbALLHbB)
  source 1..930: mol_type = other DNA
tcaatgatacttgtgagcaagagcatttgctacgccggcaacaaccttctgaaatgcagcctgcacattagggttgaaatcatgaccaagtctacgggctaggactacaacaatgacgttgccgagtaggcgaaaattttcagggtcgacgtgaagttgatcacaatgcaactcagataacttcgcaaaagtacctttcaggttgtccaaatgcttcagcccatcagagaatgattgcaatactttctttccatgagctttcacttttggatttcccataacagcatctgcattggacaaatcaccaaagctctcaaagaatctttgtgtccaggggtaaaccacaaggagcctcccaagtgcctctccaccaacttcatcaacattaacttttccccacaaccctaacactgcttccttttcctcagcactgaggtgcacacttcctccacctccagatcctccacctcctgatcctccacctcctgatcctccacctccactcctatatttgctggtgagtacagttgagacgttggcgaggaacttgtccaaactggcatgcacagaaggattaaaatcatctggatggtgggcggctaaagtgactaacaaacaatgggacagcagcttgaaatttaccggatcaactctcaatttgtgtgcatgaagatcagataatgcagaaagtgcgccgggaaggtcatccaagtgcccaacagcttttgtcaaagcatcagccaccttctgcccatgtgccttcacttgatcacttccatgtgagaggttgaaatgaggaaagtaagtctttgtcgttggaaagccaagaaacatacgctcaagagcttcagcaccgtgcgctccagcttgaccaccaacttttccccaagcagccttaacatttgctttgtctgctgccgatagaaccat

SEQ ID NO: 2 - DNA, 465 bp, synthetic construct
  misc_feature 1..465: note = porcine Myoglobin
  source 1..465: mol_type = other DNA
atggggctatcagatggtgaatggcaacttgtattgaatgtttggggaaaagttgaagctgatgttgctggacatggtcaagaagtgttaataagactcttcaaaggccaccctgaaacattagagaagtttgacaaattcaagcacctaaaatctgaagatgaaatgaaggcctccgaggacttgaagaagcatggaaacactgtcctgactgcactcggcgggatcctcaaaaagaaaggtcatcatgaagcggagttgacaccattggctcagtctcatgctaccaaacacaagattcctgtgaagtatcttgagtttattagtgaggccataattcaggttttgcaatcaaaacatcccggtgattttggtgcagatgctcaaggagcaatgagcaaagcactggagcttttcaggaatgatatggcagccaagtacaaggaacttggatttcaggggtga

SEQ ID NO: 3 - DNA, 4728 bp, synthetic construct
  misc_feature 1..4728: note = pIPTRA0:p35S+HbA-LL-HbB
  source 1..4728: mol_type = other DNA
tggcaggatatattgtggtgtaaacaaattgacgcttagacaacttaataacacattgcggacgtttttaatgtactgaattaacgccgaattgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctaattcgcttcaagacgtgctcaaatcactatttccacacccctatatttctattgcactcccttttaactgttttttattacaaaaatgccctggaaaatgcactccctttttgtgtttgtttttttgtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccgcgtaagagacaacaagtaggtacgtggagtgtcttaggtgacttacccacataacataaagtgacattaacaaacatagctaatgctcctatttgaatagtgcatatcagcataccttattacatatagataggagcaaactctagctagattgttgagcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatcccccgttcgtaaatggtgaaaattttcagaaaattgcttttgctttaaaagaaatgatttaaattgctgcaatagaagtagaatgcttgattgcttgagattcgtttgttttgtatatgttgtgttgagaattaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacctgcagactagtccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattgagctcgcccgggaaagcttcaatgatacttgtgagcaagagcatttgctacgccggcaacaaccttctgaaatgcagcctgcacattagggttgaaatcatgaccaagtctacgggctaggactacaacaatgacgttgccgagtaggcgaaaattttcagggtcgacgtgaagttgatcacaatgcaactcagataacttcgcaaaagtacctttcaggttgtccaaatgcttcagcccatcagagaatgattgcaatactttctttccatgagctttcacttttggatttcccataacagcatctgcattggacaaatcaccaaagctctcaaagaatctttgtgtccaggggtaaaccacaaggagcctcccaagtgcctctccaccaacttcatcaacattaacttttccccacaaccctaacactgcttccttttcctcagcactgaggtgcacacttcctccacctccagatcctccacctcctgatcctccacctcctgatcctccacctccactcctatatttgctggtgagtacagttgagacgttggcgaggaacttgtccaaactggcatgcacagaaggattaaaatcatctggatggtgggcggctaaagtgactaacaaacaatgggacagcagcttgaaatttaccggatcaactctcaatttgtgtgcatgaagatcagataatgcagaaagtgcgccgggaaggtcatccaagtgcccaacagcttttgtcaaagcatcagccaccttctgcccatgtgccttcacttgatcacttccatgtgagaggttgaaatgaggaaagtaagtctttgtcgttggaaagccaagaaacatacgctcaagagcttcagcaccgtgcgctccagcttgaccaccaacttttccccaagcagccttaacatttgctttgtctgctgccgatagaaccatggatcctctagtggtagatcccccgttcgtaaatggtgaaaattttcagaaaattgcttttgctttaaaagaaatgatttaaattgctgcaatagaagtagaatgcttgattgcttgagattcgtttgttttgtatatgttgtgttgagaattaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtctccggatattaccctttgttgaaaagtctcaattgccctttggtcttctgagactgtatctttgatatttttggagtagacaagtgtgtcgtgctccaccatgttgactctagagaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggagcttgagcttggatcagattgtcgtttcccgccttcagtttaaactatcagtgtttgacaggatatattggcgggtaaac

SEQ ID NO: 4 - DNA, 5649 bp, synthetic construct
  source 1..5649: mol_type = other DNA
  misc_feature 1..5649: note = pIPTRA0:p7S+HbA-LL-HbB-Arc5T
tggcaggatatattgtggtgtaaacaaattgacgcttagacaacttaataacacattgcggacgtttttaatgtactgaattaacgccgaattgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctaattcgcttcaagacgtgctcaaatcactatttccacacccctatatttctattgcactcccttttaactgttttttattacaaaaatgccctggaaaatgcactccctttttgtgtttgtttttttgtgaaacgatgttgtcaggtaatttatttgtcagtctactatggtggcccattatattaatagcaactgtcggtccaatagacgacgtcgattttctgcatttgtttaaccacgtggattttatgacattttatattagttaatttgtaaaacctacccaattaaagacctcatatgttctaaagactaatacttaatgataacaattttcttttagtgaagaaagggataattagtaaatatggaacaagggcagaagatttattaaagccgcgtaagagacaacaagtaggtacgtggagtgtcttaggtgacttacccacataacataaagtgacattaacaaacatagctaatgctcctatttgaatagtgcatatcagcataccttattacatatagataggagcaaactctagctagattgttgagcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatcccccgttcgtaaatggtgaaaattttcagaaaattgcttttgctttaaaagaaatgatttaaattgctgcaatagaagtagaatgcttgattgcttgagattcgtttgttttgtatatgttgtgttgagaattaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacctgcagactagtcactattttcagaagaagttcccaatagtagtccaaaatttttgtaacgaagggagcataatagttacatgcaaaggaaaactgccattctttagaggggatgcttgtttaagaacaaaaaatatatcactttcttttgttccaagtcattgcgtatttttttaaaaatatttgttccttcgtatatttcgagcttcaatcactttatggttctttgtattctggctttgctgtaaatcgtagctaaccttcttcctagcagaaattattaatacttgggatatttttttagaatcaagtaaattacatattaccaccacatcgagctgcttttaaattcatattacagccatataggcttgattcattttgcaaaatttccaggatattgacaacgttaacttaataatatcttgaaatattaaagctattatgattaggggtgcaaatggaccgagttggtggtgtaaatatatgagctcccattttatttattgctcccattttatttattttagtttgtgtgacagatgaacattattaggaggaaaggtataagcagggtttaactgtcacagggaaggtggttttgggagtctaaagcttcaatgatacttgtgagcaagagcatttgctacgccggcaacaaccttctgaaatgcagcctgcacattagggttgaaatcatgaccaagtctacgggctaggactacaacaatgacgttgccgagtaggcgaaaattttcagggtcgacgtgaagttgatcacaatgcaactcagataacttcgcaaaagtacctttcaggttgtccaaatgcttcagcccatcagagaatgattgcaatactttctttccatgagctttcacttttggatttcccataacagcatctgcattggacaaatcaccaaagctctcaaagaatctttgtgtccaggggtaaaccacaaggagcctcccaagtgcctctccaccaacttcatcaacattaacttttccccacaaccctaacactgcttccttttcctcagcactgaggtgcacacttcctccacctccagatcctccacctcctgatcctccacctcctgatcctccacctccactcctatatttgctggtgagtacagttgagacgttggcgaggaacttgtccaaactggcatgcacagaaggattaaaatcatctggatggtgggcggctaaagtgactaacaaacaatgggacagcagcttgaaatttaccggatcaactctcaatttgtgtgcatgaagatcagataatgcagaaagtgcgccgggaaggtcatccaagtgcccaacagcttttgtcaaagcatcagccaccttctgcccatgtgccttcacttgatcacttccatgtgagaggttgaaatgaggaaagtaagtctttgtcgttggaaagccaagaaacatacgctcaagagcttcagcaccgtgcgctccagcttgaccaccaacttttccccaagcagccttaacatttgctttgtctgctgccgatagaaccatggatccggctatcgttcgtaaatggtgaaaattttcagaaaattgcttttgctttaaaagaaatgatttaaattgctgcaatagaagtagaatgcttgattgcttgagattcgtttgttttgtatatgttgtgttgagaattgttgtgttagagagctacaatatatagagaaagatcaccgtttggctagctttaagtggctaagtgtgaacgggatgagattctcttttcttttggccgtgttggtcacaagattggacgtcgacgtgtttgaggcttgtgaaatttccagaagcaaagaaaggataaacttgagttaacatgcaatggggagaaaaacaaagggttggtgagtgtgaggggtacttggcagctgagaacaatgctttagttgcatgccatgcagatttcatccgtttgaaactctgtaaaggacgtgtggatcgagttcgcgtgagcagctagcttggcttggatttttgtcttgctatccctctactacattaatttccctacatgcaactgtttcatgacattttcgtgtatctcccaactggctagctcatattaactaaggaaaatagaattctaatggaaaatttaaataatagactttcatattttatttgtccccctcaaattttttacatttcattttttcgattttattctatttatttttttattctttttttaaaaattggcattctaaaccctatatattatatgaaataatatattttttaacaaactctttattattcatcaaaattataaatgcatgaatgaagtattatataagaagtgaaacttataaaattatgtaatttataataagtttcaagtacgcgtgttcaccaacaaaatcacgtgaaaaattgaacaaaagacgcaaaagcaagaccaagtagcctcctgagtgatgcgcttcaactgttgcaaacactaacctaaacatagacggcttctagggtgcgcaaagttgaaatgtgaggcacggtacacaagtttttttaggaccgttggatataacacttaattagttaacggtgcaaatctccaagattttagaagtggaaaaagtattgaataaaaaagtatcgcatttactgtagagcaaactcttattttaatattgttgggtcacgtgggtgtgggcttctcgactccaccggatgatgttttcaattttgatttcttttttttcctaaaggttttccatttacatttattttggttagaaaaaagaataaggctaaatgtctaaaccaacttgcattcgaacctagaactaaaataatctaccaccacgccatcatcaactttatacttttgaaaagtatttataacaatatatatattttatcaaacatgcttaattgccttaaaataaaatttataaattagttggtaatattttaacaatattaacggatttctggtaaaaaaaaataaaatcatgaatttctaaaattttaaaatcaaattttttaatgtatagattattttatctagagaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggagcttgagcttggatcagattgtcgtttcccgccttcagtttaaactatcagtgtttgacaggatatattggcgggtaaac

SEQ ID NO: 5 - DNA, 1667 bp, synthetic construct
  misc_feature 1..1667: note = 35S+TEV+myoglobincDNA+NOS
  source 1..1667: mol_type = other DNA
aataatgcggccgcatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctgaattaattctcaacacaacatatacaaaacaaacgaatctcaagcaatcaagcattctacttctattgcagcaatttaaatcatttcttttaaagcaaaagcaattttctgaaaattttcaccatttacgaacgatagatggggctatcagatggtgaatggcaacttgtattgaatgtttggggaaaagttgaagctgatgttgctggacatggtcaagaagtgttaataagactcttcaaaggccaccctgaaacattagagaagtttgacaaattcaagcacctaaaatctgaagatgaaatgaaggcctccgaggacttgaagaagcatggaaacactgtcctgactgcactcggcgggatcctcaaaaagaaaggtcatcatgaagcggagttgacaccattggctcagtctcatgctaccaaacacaagattcctgtgaagtatcttgagtttattagtgaggccataattcaggttttgcaatcaaaacatcccggtgattttggtgcagatgctcaaggagcaatgagcaaagcactggagcttttcaggaatgatatggcagccaagtacaaggaacttggatttcaggggtgagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcggccgcttatta

SEQ ID NO: 6 - DNA, 3232 bp, synthetic construct
  misc_feature 1..3232: note = p7S+TEV+myoglobincDNA+arc5+Rb7MAR
  source 1..3232: mol_type = other DNA
aaatttgcggccgcggtacttggcagctgagaacaatgctttagttgcatgccatgcagatttcatccgtttgaaactctgtaaaggacgtgtggatcgagttcgcgtgagcagctagcttggcttggatttttgtcttgctatccctctactacattaatttccctacatgcaactgtttcatgacattttcgtgtatctcccaactggctagctcatattaactaaggaaaatagaattctaatggaaaatttaaataatagactttcatattttatttgtccccctcaaattttttacatttcattttttcgattttattctatttatttttttattctttttttaaaaattggcattctaaaccctatatattatatgaaataatatattttttaacaaactctttattattcatcaaaattataaatgcatgaatgaagtattatataagaagtgaaacttataaaattatgtaatttataataagtttcaagtacgcgtgttcaccaacaaaatcacgtgaaaaattgaacaaaagacgcaaaagcaagaccaagtagcctcctgagtgatgcgcttcaactgttgcaaacactaacctaaacatagacggcttctagggtgcgcaaagttgaaatgtgaggcacggtacacaagtttttttaggaccgttggatataacacttaattagttaacggtgcaaatctccaagattttagaagtggaaaaagtattgaataaaaaagtatcgcatttactgtagagcaaactcttattttaatattgttgggtcacgtgggtgtgggcttctcgactccaccggatgatgttttcaattttgatttcttttttttgctaaaggttttccatttacatttattttggttagaaaaaagaataaggctaaatgcctaaaccaacttgcattcgaacctagaactaaaataatctaccaccacgccatcatcaactttatacttttgaaaagtatttataacaatatatatattttatcaaacatgcttaattgccttaaaataaaatttataaattagttggtaatattttaacaatattaacaaatttctggtaaaaaaattaaaatcatgaatttctaaaattttaaaatcaaattttttaatgtataggttattttaatttaatttatattgttaaaaatgggttattacaatttaattaatctctaattaaaatatatatgaggatattagttttattctaatactattgtaattcccattcatatgaatatatatatatgagattgtttgataaaataatctcaataaatgtttcaactatgcaaagcataatgagcatctaacttatttacaatttacaataataaatcatgatcaaaacaaacatcaacactaactcgttattagtatgttatcaacaaatgtaaaggtgaagctactctagcttaagtcaaagttaaattattctgatcagaaactctttgaaaatatacctaataaaactcaagaaaatacacacctaatataaacatatgttgaacatctttatacatgtgcatcttcaaacccctattcattccatctattgtgtctccaagaaggagatccaacctataagtcatttcctcccatgtacgtcatgtataggatcattacagtcacagccacatgtgttatatgcaaggatttaattagtagtgtaaaaacttgagtggaatccttcaaggattagaaagagaaactaaggtaaattaagagatttactgaatcaatataaatcttttgtttttactcgagcttctattatacttgttttatcttagttgcttctatcttactttcgatgtttaaattttgagaaaaaaatccttttgtgaaaaacctttttaaaaagttgtttgacgctaccgtacagagcatccatttttaatttgtggtcaaatttgctttgtgaaaatcttcatcttacaaaaaaaaaaccttaatttaaatctcatctaacatattgaattaattctcaacacaacatatacaaaacaaacgaatctcaagcaatcaagcattctacttctattgcagcaatttaaatcatttcttttaaagcaaaagcaattttctgaaaattttcaccatttacgaacgatagatggggctatcagatggtgaatggcaacttgtattgaatgtttggggaaaagttgaagctgatgttgctggacatggtcaagaagtgttaataagactcttcaaaggccaccctgaaacattagagaagtttgacaaattcaagcacctaaaatctgaagatgaaatgaaggcctccgaggacttgaagaagcatggaaacactgtcctgactgcactcggcgggatcctcaaaaagaaaggtcatcatgaagcggagttgacaccattggctcagtctcatgctaccaaacacaagattcctgtgaagtatcttgagtttattagtgaggccataattcaggttttgcaatcaaaacatcccggtgattttggtgcagatgctcaaggagcaatgagcaaagcactggagcttttcaggaatgatatggcagccaagtacaaggaacttggatttcaggggtgatagactcccaaaaccaccttccctgtgacagttaaaccctgcttatacctttcctcctaataatgttcatctgtcacacaaactaaaataaataaaatgggagcaataaataaaatgggagctcatatatttacaccaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggcggccgcttaata

SEQ ID NO: 7 - DNA, 2635 bp, synthetic construct
  misc_feature 1..2635: note = PPhas+myoglobincDNA+arc5+Rb7MAR
  source 1..2635: mol_type = other DNA
aaatttgcggccgccattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataaagatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatcatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataacatttatggtggactaattttcatatatttcttattgcttttaccttttcttggtatgtaagtccgtaactagaattacagtgggttgccatggcactctgtggtcttttggttcatgcatgggtcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctactatggggctatcagatggtgaatggcaacttgtattgaatgtttggggaaaagttgaagctgatgttgctggacatggtcaagaagtgttaataagactcttcaaaggccaccctgaaacattagagaagtttgacaaattcaagcacctaaaatctgaagatgaaatgaaggcctccgaggacttgaagaagcatggaaacactgtcctgactgcactcggcgggatcctcaaaaagaaaggtcatcatgaagcggagttgacaccattggctcagtctcatgctaccaaacacaagattcctgtgaagtatcttgagtttattagtgaggccataattcaggttttgcaatcaaaacatcccggtgattttggtgcagatgctcaaggagcaatgagcaaagcactggagcttttcaggaatgatatggcagccaagtacaaggaacttggatttcaggggtgatagactcccaaaaccaccttccctgtgacagttaaaccctgcttatacctttcctcctaataatgttcatctgtcacacaaactaaaataaataaaatgggagcaataaataaaatgggagctcatatatttacaccaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggcggccgcttaaat

SEQ ID NO: 8 - DNA, 781 bp, synthetic construct
  misc_feature 1..781: note = 2x 35S promoter
  source 1..781: mol_type = other DNA
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctct

SEQ ID NO: 9 - DNA, 139 bp, synthetic construct
  misc_feature 1..139: note = TEV
  source 1..139: mol_type = other DNA
gaattaattctcaacacaacatatacaaaacaaacgaatctcaagcaatcaagcattctacttctattgcagcaatttaaatcatttcttttaaagcaaaagcaattttctgaaaattttcaccatttacgaacgatag

SEQ ID NO: 10 - DNA, 253 bp, synthetic construct
  misc_feature 1..253: note = NOS terminator
  source 1..253: mol_type = other DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 11 - DNA, 2000 bp, synthetic construct
  misc_feature 1..2000: note = 7S promoter
  source 1..2000: mol_type = other DNA
ggtacttggcagctgagaacaatgctttagttgcatgccatgcagatttcatccgtttgaaactctgtaaaggacgtgtggatcgagttcgcgtgagcagctagcttggcttggatttttgtcttgctatccctctactacattaatttccctacatgcaactgtttcatgacattttcgtgtatctcccaactggctagctcatattaactaaggaaaatagaattctaatggaaaatttaaataatagactttcatattttatttgtccccctcaaattttttacatttcattttttcgattttattctatttatttttttattctttttttaaaaattggcattctaaaccctatatattatatgaaataatatattttttaacaaactctttattattcatcaaaattataaatgcatgaatgaagtattatataagaagtgaaacttataaaattatgtaatttataataagtttcaagtacgcgtgttcaccaacaaaatcacgtgaaaaattgaacaaaagacgcaaaagcaagaccaagtagcctcctgagtgatgcgcttcaactgttgcaaacactaacctaaacatagacggcttctagggtgcgcaaagttgaaatgtgaggcacggtacacaagtttttttaggaccgttggatataacacttaattagttaacggtgcaaatctccaagattttagaagtggaaaaagtattgaataaaaaagtatcgcatttactgtagagcaaactcttattttaatattgttgggtcacgtgggtgtgggcttctcgactccaccggatgatgttttcaattttgatttcttttttttgctaaaggttttccatttacatttattttggttagaaaaaagaataaggctaaatgcctaaaccaacttgcattcgaacctagaactaaaataatctaccaccacgccatcatcaactttatacttttgaaaagtatttataacaatatatatattttatcaaacatgcttaattgccttaaaataaaatttataaattagttggtaatattttaacaatattaacaaatttctggtaaaaaaattaaaatcatgaatttctaaaattttaaaatcaaattttttaatgtataggttattttaatttaatttatattgttaaaaatgggttattacaatttaattaatctctaattaaaatatatatgaggatattagttttattctaatactattgtaattcccattcatatgaatatatatatatgagattgtttgataaaataatctcaataaatgtttcaactatgcaaagcataatgagcatctaacttatttacaatttacaataataaatcatgatcaaaacaaacatcaacactaactcgttattagtatgttatcaacaaatgtaaaggtgaagctactctagcttaagtcaaagttaaattattctgatcagaaactctttgaaaatatacctaataaaactcaagaaaatacacacctaatataaacatatgttgaacatctttatacatgtgcatcttcaaacccctattcattccatctattgtgtctccaagaaggagatccaacctataagtcatttcctcccatgtacgtcatgtataggatcattacagtcacagccacatgtgttatatgcaaggatttaattagtagtgtaaaaacttgagtggaatccttcaaggattagaaagagaaactaaggtaaattaagagatttactgaatcaatataaatcttttgtttttactcgagcttctattatacttgttttatcttagttgcttctatcttactttcgatgtttaaattttgagaaaaaaatccttttgtgaaaaacctttttaaaaagttgtttgacgctaccgtacagagcatccatttttaatttgtggtcaaatttgctttgtgaaaatcttcatcttacaaaaaaaaaaccttaatttaaatctcatctaacatatt

SEQ ID NO: 12 - DNA, 138 bp, synthetic construct
  misc_feature 1..138: note = Arc5 Terminator
  source 1..138: mol_type = other DNA
tagactcccaaaaccaccttccctgtgacagttaaaccctgcttatacctttcctcctaataatgttcatctgtcacacaaactaaaataaataaaatgggagcaataaataaaatgggagctcatatatttacacca

SEQ ID NO: 13 - DNA, 462 bp, synthetic construct
  misc_feature 1..462: note = Rb7 Mar
  source 1..462: mol_type = other DNA
ccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtg

SEQ ID NO: 14 - DNA, 1542 bp, synthetic construct
  misc_feature 1..1542: note = Phas promoter
  source 1..1542: mol_type = other DNA
cattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataaagatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatcatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataacatttatggtggactaattttcatatatttcttattgcttttaccttttcttggtatgtaagtccgtaactagaattacagtgggttgccatggcactctgtggtcttttggttcatgcatgggtcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctact